(19) United States
(12) Patent Application Publication
(10) Pub. No.: US 2011/0144047 A1
(43) Pub. Date: Jun. 16, 2011

(54) COMBINED METHOD FOR PREDICTING THE RESPONSE TO AN ANTI-CANCER THERAPY

(75) Inventors: Nils Aage Brünner, Hellerup (DK); Kristen Vang Nielsen, Bronshoj (DK)

(73) Assignee: DAKO DENMARK A/S, Glostrup (DK)

(21) Appl. No.: 12/993,372

(22) PCT Filed: May 20, 2009
(86) PCT No.: PCT/DK2009/050116

§ 371 Date: Jan. 31, 2011

Related U.S. Application Data
(60) Provisional application No. 61/054,944, filed on May 21, 2008.

(30) Foreign Application Priority Data
May 20, 2008 (DK) PA 2008 00696

(51) Int. Cl. A61K 31/704; A61P 35/00; C12Q 1/68

(52) U.S. Cl. 514/34; 435/6.18

(57) ABSTRACT

The invention provides methods for predicting the response to a topoisomerase Ilα inhibitor therapy in an individual having cancer, wherein the methods comprise the steps of determining TIMP-I DNA aberration/TIMP-1 protein aberration in combination with determining DNA aberration in TOP2A/HER2 amplicon on chromosome 17q21 including TOP2A and HER2 or aberrations of TOP2A and ErbB2 protein expression. Further provided are methods of treating cancer by using said topoisomerase Ilα inhibitor therapy. The invention also comprises a kit for the application of the methods for predicting the response to a topoisomerase Ilα inhibitor therapy in an individual having cancer.

### TECHNICAL FIELD OF THE INVENTION

The present invention relates to the field of anti-cancer therapy. In particular the present invention relates to a method for predicting the response to various types of anti-cancer therapies. In particular the present invention relates to improvement in therapy of individuals suffering from cancer.

### BACKGROUND OF THE INVENTION

### Tissue Inhibitor of Metalloprotease-1 (TIMP-1)

Tissue Inhibitor of Metalloprotease-1 (TIMP-1) is one out a family of four endogenous inhibitors of matrix metalloproteases (MMPs) and the gene is located on the x-chromosome. TIMP-1 is a 25 kDa protein which binds most MMPs with a 1:1 stochiometry. TIMP-1 is present in various tissues and body fluids and is stored in α-granules of platelets and released upon activation. While the main function of TIMP-1 is supposed to be MMP inhibition, some alternative functions of TIMP-1 have been described, e.g. inhibition of apoptosis and regulation of cell growth and angiogenesis. In addition, some studies have suggested that TIMP-1 may also play a role in the early processes leading to the malignant phenotype.

The present inventors have described that measurement of plasma TIMP-1 gives high specificity and high sensitivity in the detection of early stage colorectal cancer. In addition, the present inventor has shown that measurement of plasma TIMP-1 levels in preoperative or postoperative samples yields strong and stage independent prognostic information in patients with early stage colorectal cancer. By measuring TIMP-1 protein in primary breast cancer tissue the inventors of the present invention and others have shown that high tumour tissue total TIMP-1 levels are associated with shorter patient survival.

A role for TIMP-1 in the regulation of apoptosis has been reported and two possible ways for this to happen have been suggested. Both of these support the idea that TIMP-1 inhibits apoptosis.

First, proteolytic degradation of the extracellular matrix leads to loss of differentiation and to apoptosis in mammary epithelial cells both in vitro and in vivo. This indicates that the integrity of the extracellular matrix and the protection of cell-matrix interactions are crucial factors in assuring survival of mammary epithelium. Through the inhibition of MMPs, TIMP-1 is capable of inhibiting degradation of extracellular matrix, thereby possibly inhibiting apoptosis. By crossing mice that over-expressed MMP-3 in the mammary gland with TIMP-1 transgenic mice, Alexander and co-workers demonstrated such apoptosis-inhibitory effect of TIMP-1 observing that apoptosis of the mammary epithelium induced by MMP-3 was reduced by TIMP-1. The mere disintegration of the basement membrane could be responsible for apoptosis induced by proteolytic activity but it has also been speculated that integrin-mediated signalling plays a part.

Second, an apoptosis-inhibitory effect of TIMP-1 that occurs independently of MMP-inhibition has also been demonstrated. In human breast epithelial cells, an ability of endogenous TIMP-1 to inhibit apoptosis induced by abolition of cell adhesion has been demonstrated. This indicates that TIMP-1 is capable of rescuing cells from apoptosis without stabilising extracellular matrix and cell-matrix interactions. The independence of MMP-inhibition in inhibiting apoptosis is supported by the fact that reduced and alkylated TIMP-1, which has lost all MMP-inhibitory effect, still effectively inhibits apoptosis in Burkitt's lymphoma cell lines. The mechanism for this apoptosis-inhibitory effect is not known at present, but different suggestions have been made regarding signalling pathways possibly regulated by TIMP-1. Over-expression of TIMP-1 in human breast epithelial cells is associated with more efficient activation and constitutive activity of focal adhesion kinase (FAK)—a kinase that is normally involved in signalling cell survival. Also, up-regulation of TIMP-1 protein expression in Burkitt's lymphoma cells increased the expression of the anti-apoptotic protein Bcl-X_{L}. It was speculated that the modulation of cell signalling is mediated via interaction of TIMP-1 with a cell surface receptor as the anti-apoptotic effect of TIMP-1 in Burkitt's lymphoma cells was abolished by the neutralisation of secreted TIMP-1 by monoclonal antibodies. This view is further supported by a study that demonstrates binding of TIMP-1 to CD63 located on the surface of breast epithelial cells.

Accordingly, TIMP-1 appears to be capable of inhibiting apoptosis via two different mechanisms. Through inhibition of MMPs, TIMP-1 stabilises extracellular matrix and cell-matrix interactions thereby inhibiting apoptosis induced by disintegration of the extracellular matrix. However, TIMP-1 also inhibits apoptosis via a mechanism that is not dependent of its ability to inhibit proteolytic degradation of the extracellular matrix. This latter mechanism may be mediated by the interaction of TIMP-1 with a receptor on the cell surface regulating intracellular signalling pathways involved in apoptosis.

Two clinical studies by the inventors have suggested predictive value of TIMP-1 protein measurements (Schrohl et al., 2006 and Sorensen et al. 2007). In the study by Schrohl et al, TIMP-1 protein was measured in breast cancer extracts using ELISA. The authors describe that high TIMP-1 protein levels are associated with lack of response to chemotherapy in patients with metastatic breast cancer. In the study by Sorensen et al., the authors describe the predictive value of plasma TIMP-1 protein levels determined by ELISA. The results of this study shows that patients with metastatic colorectal cancer and high plasma TIMP-1 levels have a decreased objective response rate and a decreased survival following treatment with irinotecan based chemotherapy as compared to patients with low TIMP-1 protein levels in plasma. These two studies are in line with preclinical data generated by the inventor showing increased sensitivity to chemotherapy in cancer cells made deficient for the TIMP-1 gene (Davidsen et al. 2006).

Topoisomerase llα

The TOP2A gene is located on chromosome 17q21, in the same amplicon as HER2, where it codes for the enzyme topoisomerase lla. This enzyme is involved in the regulation of DNA topology and is important for the integrity of the genetic material during transcription, replication and recombination processes. During these processes topoisomerase lla catalyzes the breakage and reunion of double stranded DNA. The expression of the topoisomerase lla is cell cycle dependent with markedly higher levels in exponentially growing than in quiescent cell lines. It has been shown that the amount of the enzyme correlates with cell proliferation The predominant genetic mechanism for oncogene activation is through amplification of genes that leads to protein over-expression and provides the tumor with selective growth advantages. Amplification of the TOP2A gene has been reported in 7-14% of patients with breast cancers and deletions with a similar frequency. In comparison, the HER2 oncogene is amplified in 20-30% of the breast cancer patients (Harris et al. 2002).

Topoisomerase IIa is the pharmacological target of anthracyclines and several studies have shown that TOP2A gene aberrations, especially amplification, are predictive to the response to anthracycline based chemotherapy in patients with primary breast cancer (Park et al. 2003, Press et al. 2005, Tanner et al. 2005, Knoop et al. 2005). Fewer data are available with respect to patients with TOP2A deletions but a better treatment outcome for this group of patients has been observed as well. However, analysing for TOP2A amplifications or deletions will only identify approximately 20% of the breast cancer patient population as being anthracycline sensitive. This number should be seen in the context of the estimated 50% of high-risk breast cancer patients having benefit from adjuvant anthracyclines.

In one study a significant association between TOP2A amplification and topoisomerase lla protein was found. Over-expression of topoisomerase lla protein was present in 93% of the cases with amplification of TOP2A. However, the other way around, only 20% of cases with over-expression had amplification. Other studies have failed to show a similar correlation (Petit et al. 2004, Mueller et al. 2004, Durbecq et al. 2004).

Jorgensen et al. discloses a review of the pharmadiagnostic possibilities with respect to therapy selection in breast cancer including the predictive value of TOP2A and HER-2 gene aberrations. The review states that a number of clinical studies have shown that patients who have tumours with TOP2A gene aberrations, especially amplifications, experience a significantly better effect from anthracycline-based chemotherapy that patients with normal TOP2A gene status. WO 2007/112746 discloses a method for performing a prognostic evaluation for high-risk breast cancer patients using TOP2A gene aberrations. The method for performing the prognostic evaluation comprises the steps of determining the status of an aberration of the TOP2A gene and estimating the probability of either recurrence-free survival or of overall survival of the patient at a later time based upon a predefined Hazard Ratio or a pre-determined Kaplan-Meier plot corresponding to the determined status. It is well known that the term prognosis covers the fate of the disease in an untreated patient and prognostic evaluation is thus not the same as predictive evaluation, the latter term covering the likelihood of a patient to benefit from a specific treatment.

### SUMMARY OF THE INVENTION

Thus, as it appears from the above, there is a need in the art for additional predictive markers that can identify additional patients that will benefit from anthracycline treatment.

Thus, an object of the present invention relates to improvement of patient selection for treatment with a topoisomerase IIα inhibitor therapy such as a topoisomerase IIα inhibitor therapy comprising an anthracycline.

In particular, it is an object of the present invention to provide a method that solves the above mentioned problems of the prior art with identifying a relevant proportion of breast cancer patients in whom topoisomerase IIα inhibitor therapy will have a high likelihood of being effective.

Thus, one aspect of the invention relates to a method for predicting the response to a topoisomerase IIα inhibitor therapy in an individual having cancer, said method comprising the steps of:
a. determining in a sample obtained from said individual, the absence of TIMP-1 protein in tumour cells comprised in said sample or presence of a TIMP-1 DNA aberration in the tumour cells of said sample, and
b. determining the presence of any chromosomal DNA aberration in the TOP2A/HER2 amplicon on chromosome 17q21 or aberrant protein expression of a gene comprised in said amplicon
c. classifying the individual as having a high likelihood of responding to a topoisomerase IIα inhibitor therapy if a chromosomal DNA aberration in the TOP2A/HER2 amplicon on chromosome 17q21 is present and/or the protein expression of the gene comprised in said amplicon is aberrant in said tumour cells and/or if the tumour cells are absent of TIMP-1 protein and/or if said tumour cells comprise said TIMP-1 DNA aberration on either or both of the alleles of the TIMP-1 gene, and
d. classifying the individual as having a low likelihood of responding to a topoisomerase IIα inhibitor therapy if no chromosomal DNA aberration in the TOP2A/HER2 amplicon is present or no protein encoded by any gene comprised in said amplicon is aberrantly expressed in the tumour cells and if TIMP-1 protein is present in the tumour cells and/or if neither of the TIMP-1 alleles comprise said TIMP-1 DNA aberration.

A second aspect relates to a method for predicting the response to a topoisomerase IIα inhibitor therapy in an individual having cancer, said method comprising the steps of:
a. determining in a sample obtained from said individual, the absence of TIMP-1 protein in tumour cells comprised in said sample, and
b. determining the presence of any TOP2A DNA aberration in the tumour cells of said sample
c. classifying the individual as having a high likelihood of responding to a topoisomerase IIα inhibitor therapy if a TOP2A DNA aberration is present and/or if the tumour cells are absent of TIMP-1 protein, and
d. classifying the individual as having a low likelihood of responding to a topoisomerase IIα inhibitor therapy if no TOP2A DNA aberration is present and if TIMP-1 protein is present in the tumour cells.

In one embodiment the cancer is selected from the group consisting of breast cancer, sarcomas, ovarian cancer, and lung cancer. In a preferred embodiment the cancer is breast cancer.

Another aspect of the present invention relates to a method of treating cancer in an individual comprising
a. predicting the response to an topoisomerase IIα inhibitor therapy according to any of the preceeding claims, and
b. selecting a topoisomerase IIα inhibitor therapy to which said individual has a high likelihood of responding to,
c. subjecting to said individual said topoisomerase IIα inhibitor therapy.

In one embodiment, the topoisomerase IIα inhibitor used in said method of treatment is an anthracyclines such as 4-Epirubricin, which in a further embodiment is used in combination with cyclophosphamide and 5-fluorouracil or a taxane.

Yet another aspect of the present invention is to provide a kit for predicting the response to a topoisomerase IIα inhibitor therapy comprising
a. reagents suitable for the determination of a chromosomal DNA aberration in the TOP2A/HER2 amplicon such TOP2A or HER2 DNA aberrations in a biological sample, and
b. reagents suitable for the determination of a TIMP-1 DNA aberration or determining the level of a TIMP-1 protein in a biological sample.

### DETAILED DESCRIPTION OF THE INVENTION

It is well known that measurement of TOP2A DNA aberrations in beast cancer cells can predict benefit from adjuvant anthracycline containing chemotherapeutic drug regimes (Knoop et al JCO 2005). However, since only approximately 20% of primary breast cancer patients will display TOP2A DNA aberrations in their tumor cells, this method only allows for identification of 20% of the breast cancer population who have an increased likelihood of benefit from adjuvant anthracyline treatment. This number should bee seen in the light of approximately 50% of primary breast cancer patient knowing to benefit from anthracycline treatment.

A number of other potential predictive markers have been combined with the TOP2A DNA aberration measurements, e.g. HER2, but no additive effect between these two biomarkers has been seen with regard to disease free survival or overall survival in the identified subgroup (Knoop et al., JCO 2005). Thus, at present, there is no biomarker (DNA, mRNA or protein) that has shown superior prediction to benefit from anthracycline treatment when combined with TOP2A DNA aberrations than TOP2A DNA aberration measurements alone. This is further supported by O'Malley et al. 2009 that shows that combining TOP2A and HER DNA measurements does not improve the predictive value above what can be obtained by each of the two markers.

It has previously been reported that high tumour extract protein levels of TIMP-1 protein in primary tumours derived from patients with metastatic breast cancer is associated with decreased likelihood of obtaining an objective response to chemotherapy (both anthracycline containing and not anthracycline containing drug combinations). This likelihood decreases with increasing expression of TIMP-1. The TIMP-1 protein was measured by ELISA (Schrohl et al., Clin Cancer Res 2006).

Sørensen et al. Clin Cancer Res 2007 pertains to the effect of chemotherapy on patients having metastatic colorectal cancer. In this study TIMP-1 is combined with CEA—the study shows that the combination with CEA does not provide any additive effect.

The inventors disclose for the first time that lack of TIMP-1 immunoreactivity in breast cancer cells is associated with likelihood of benefit from adjuvant anthracyline treatment but not non-anthracycline containing chemotherapy. In a retrospective study including 649 patients with high risk breast cancer, the inventors show that patients who's tumor cells lack TIMP-1 immunoreactivity are those who benefit the most from adjuvant anthracycline treatment as compared with patients who's tumor cells lack TIMP-1 immunoreactivity and who receive adjuvant treatment with a non-anthracycline containing chemotherapy regimen (CMF) or patients who's tumor cells show TIMP-1 immunoreactivity and who receive adjuvant therapy with either anthracycline or non-anthracycline containing chemotherapy.

Thus, the present invention allows for the identification of high risk breast cancer patients with a high likelihood of benefit from adjuvant anthracycline treatment: Lack of TIMP-1 immunoreactivity in the breast cancer cells identifies app 20% of the patients who have a high likelihood of benefit from adjuvant anthracycline treatment. In practical terms, by TIMP-1 immunohistochemistry, it will be possible to identify app 20% of the patients scheduled for adjuvant treatment who will have a high likelihood of benefit from the treatment. On other hand, TIMP-1 immunohistochemistry also allows for the identification of app 80% of the patients who are scheduled for adjuvant anthracycline containing treatment who would do equally well by treatment with the much less toxic CMF. Alternatively, these 80% of the patients could be treated with any other active drug than anthracyclines, used in adjuvant treatment of breast cancer e.g. taxanes, Methotrexate, Cyclophosphamide, 5 Fluorouracil and gemcitabine (Example 1).

The inventors report for the first time that the combination of TIMP-1 breast cancer cell immunoreactivity measurements and TOP2A DNA aberration measurements in the same tumor cells yields additive predictive value, i.e. each of the two tests identify approximately 20% of patients having a high likelihood of obtaining benefit from adjuvant anthracyline containing chemotherapy, and since there is only 4% overlap between the two patient populations, the effect of the combined assay is additive.

Thus, the present invention allows for the identification of almost double as many breast cancer patients with a high likelihood of benefit from adjuvant anthracycline treatment: TOP2A DNA aberration measurements identifies app 20% and lack of TIMP-1 immunoreactivity assay identifies app 20% of the patients who have a high likelihood of benefit from adjuvant anthracycline treatment. In practical terms, by the combined assay, it will be possible to identify app 40% of the patients scheduled for adjuvant treatment who will have a high likelihood of benefit from the treatment. On other hand, the combined assay also allows for the identification of app 60% of the patients who are scheduled for adjuvant anthracycline containing treatment who would do equally well by treatment with the much less toxic CMF. Alternatively, these 60% of the patients could be treated with any other active drug than anthracyclines, used in adjuvant treatment of breast cancer e.g. taxanes, Methotrexate, Cyclophosphamide, 5 Fluorouracil and gemcitabine (Example 3).

The present inventors recently found TIMP-1 gene aberrations (deletions and amplifications) in breast cancer cells.

The present application discloses a study of TOP2A gene aberrations and TIMP-1 protein tumor cell content in 641 breast cancer patients who were randomized to receive adjuvant treatment with either Cyclophosfamide, Methotrexate and 5-fluorouracil (CMF) or Cyclophosfamide, 4-Epirubricin and 5-Fluorouracil (CEF). Endpoint was disease free survival (DFS). As previously reported on this patient cohort (Knoop et al), TOP2A aberrations were predictive for benefit (increased DFS) from CEF but not from CMF. When performing TIMP-1 immunohistochemistry using the VT7 anti TIMP-1 monoclonal antibody the inventor found that approximately 80% of the patients showed TIMP-1 immunoreactivity in the tumor cells. The remaining 20% of the tumors were absent of TIMP-1 tumor cell immunoreactivity. When performing statistical survival analyses, it was found that lack of TIMP-1 immunoreactivity in the tumor cells was significantly associated to the end-point: DFS, with a longer DFS of the patients. In contrast, no differences in DFS in relation to TIMP-1 immunoreactivity were observed in patients receiving CMF.

When combining the results of the TOP2A and TIMP-1 analyses, it was seen that these two biomarkers were additive in predicting response to CEF while no effect of the combination of these two biomarkers were observed in the CMF treated patients. The additive effect was based on the fact that there was only a very little overlap between the patients having TOP2A gene aberrations and patients lacking TIMP-1 immunoreactivity in their tumor cells (4% overlap). Since the two groups were almost identical in size, the combination of these two biomarkers doubled the number of patients that could be predicted as CEF responders without loosing power of the predictive value of each of the biomarkers.

This means that by the use of the combined TOP2A and TIMP-1 test, patients who will benefit the most from adjuvant anthracycline treatment can be identified. On the other hand, the combined test can also be used to identify the approximately 60% of patients who would do equally well by receiving a non-anthracycline containing chemotherapy regimens or perhaps even better by receiving another drug combination, e.g. combinations including taxanes. This invention should be seen in the light of lack of additive effect when combining TOP2A with HER2 (Knoop et al 2005) and lack of additive effect when combing TIMP with CEA in colorectal cancer drug prediction (Sørensen et al., 2007)

In order to extend the available methods for performing prediction of therapy effectiveness for breast cancer patients, beyond what is presently available in the art, novel methods for performing such prediction are herein disclosed, wherein the prediction is based upon the determined status of TOP2A gene aberrations (wherein the term “status” refers to the presence or absence of an aberration and, if an aberration is present, the type—amplification or deletion—of the aberration) or TOP2A protein together with determination of TIMP-1 protein or TIMP-1 DNA aberrations in the tumor cells. Embodiments in accordance with the invention may comprise the steps of determining the status of an aberration of the TOP2A gene together with the TIMP-1 gene or protein status in a breast cancer tissue sample taken from a patient; and based on the results of such testing one can estimate for the individual patient the likelihood of obtaining benefit from anthracycline containing chemotherapy as compared to non-anthracycline containing chemotherapy.

For example, patients with TOP2A aberrations and/or absence of TIMP-1 immunoreactivity in the cancer cells should be offered chemotherapy containing anthracyclines, while the remaining patients will do equally well receiving anthracyclines or non-anthracyclines. Based on the severe toxicity of anthracyclines, it would be correct to offer the latter patients a non-anthracycline containing chemotherapy regimen.

The presently presented methods thus rely on the surprising discovery that it is possible to almost double the predictive value of TOP2A determinations in breast cancer patients by adding the analysis of TIMP-1 tumor cell immune reactivity in the breast cancer cells.

The invention is based on a method for predicting whether a cancer patient will benefit from an anti-cancer therapy, where the efficiency of said anti-cancer therapy depends on tumour tissue TOP2A gene aberrations in the tumor cells combined with absence of TIMP-1 immunoreactivity in the cancer cells, the method comprising determining whether cells from tumour tissue in the patient have TOP2A gene aberrations or lack TIMP-1 immunoreactivity, and establishing that the patient most likely will benefit from a specific anti-cancer therapy if TOP2A DNA aberrations or lack of TIMP-1 immunoreactivity is observed.

In the present application the anti-cancer therapy preferably refers to a topoisomerase II inhibitor therapy.

The prediction method of the invention preferably comprises that the determination of whether cells from tumour tissues in the patient have TOP2A gene aberrations and/or lack TIMP-1 immunoreactivity is performed by measuring on a sample selected from the group consisting of a tumour tissue sample, a blood sample, a plasma sample, a serum sample, a urine sample, a faeces sample, a saliva sample, and a sample of serous liquid from the thoracic and abdominal cavity. The method of measuring is conveniently performed by means of DNA level measurement, mRNA level measurement such as in situ hybridization, Northern blotting, QRT-PCR, and differential display, and protein level measurement, such as Western blotting, immunohistochemistry, immunocytochemisty, ELISA, and RIA.

One can perform a retrospective/prospective clinical trial, in order to establish the threshold level for TIMP-1 protein so as to determine resistance/sensitivity to topoisomerase IIα inhibitor treatment of the individual patient.

Retrospectively, stored tumour tissue or blood or urine, or saliva or any other body fluid is obtained from patients who have experienced recurrence of their cancer disease and of whom it is known how they responded to the particular anti-cancer treatment. In the case of tumour tissue extracts, the tissue is homogenized and the level of TIMP-1 protein is measured in each individual patient sample. In the case of body fluids, the sample may be diluted and subsequently, the concentration of TIMP-1 protein is determined by one of the methods discussed herein. In the case of formalin fixed paraffin embedded tumor tissue, conventional immunohistochemistry can be performed either on the primary tumor or on tissue obtained from metastatic lesions.

Accordingly, one aspect of the present invention relates to a method for predicting the response to a topoisomerase IIα inhibitor therapy in an individual having cancer, said method comprising the steps of:
a) determining in a sample obtained from said individual, the absence of TIMP-1 protein in tumour cells comprised in said sample or presence of a TIMP-1 DNA aberration in the tumour cells of said sample, and
b) determining the presence of any chromosomal DNA aberration in the TOP2A/HER2 amplicon on chromosome 17q21 or aberrant protein expression of a gene comprised in said amplicon
c) classifying the individual as having a high likelihood of responding to a topoisomerase IIα inhibitor therapy if a chromosomal DNA aberration in the TOP2A/HER2 amplicon on chromosome 17q21 is present and/or the protein expression of the gene comprised in said amplicon is aberrant in said tumour cells and/or if the tumour cells are absent of TIMP-1 protein and/or if said tumour cells comprise said TIMP-1 DNA aberration on either or both of the alleles of the TIMP-1 gene, and
d) classifying the individual as having a low likelihood of responding to a topoisomerase IIα inhibitor therapy if no chromosomal DNA aberration in the TOP2A/HER2 amplicon is present or no protein encoded by any gene comprised in said amplicon is aberrantly expressed in the tumour cells and if TIMP-1 protein is present in the tumour cells and/or if neither of the TIMP-1 alleles comprise said TIMP-1 DNA aberration.

The TOP2A/HER2 amplicon on chromosome 17q21 referred to above comprises the TOP2A and HER2 genes.

Thus, one embodiment according to the invention, concerns predicting the response to a topoisomerase IIα inhibitor therapy in an individual having cancer, wherein the chromosomal DNA aberration in the TOP2A/HER2 amplicon on chromosome 17q21 is a TOP2A DNA aberration, and the protein expression of the gene comprised in said amplicon is topoisomerase IIa expression.

Another embodiment according to the invention concerns predicting the response to a topoisomerase IIα inhibitor therapy in an individual having cancer, wherein the chromosomal DNA aberration in the TOP2A/HER2 amplicon on chromosome 17q21 is a HER2 DNA aberration, and the protein expression of the gene comprised in said amplicon is ErbB2 expression.

In a preferred embodiment said method comprising the steps of:
a. determining in a sample obtained from said individual, the absence of TIMP-1 protein in tumour cells comprised in said sample, and
b. determining the presence of any TOP2A DNA aberration in the tumour cells of said sample
c. classifying the individual as having a high likelihood of responding to a topoisomerase IIα inhibitor therapy if a TOP2A DNA aberration is present and/or if the tumour cells are absent of TIMP-1 protein, and
d. classifying the individual as having a low likelihood of responding to a topoisomerase IIα inhibitor therapy if no TOP2A DNA aberration is present and if TIMP-1 protein is present in the tumour cells.

One embodiment of the present invention is a method for predicting the response to a topoisomerase IIα inhibitor therapy in an individual having cancer, said method comprising the steps of:
a. determining in a sample obtained from said individual, the absence of TIMP-1 protein in tumour cells comprised in said sample, and
b. determining the presence of any HER2 DNA aberration in the tumour cells of said sample
c. classifying the individual as having a high likelihood of responding to a topoisomerase IIα inhibitor therapy if a HER2 DNA aberration is present and/or if the tumour cells are absent of TIMP-1 protein, and
d. classifying the individual as having a low likelihood of responding to a topoisomerase IIα inhibitor therapy if no HER2 DNA aberration is present and if TIMP-1 protein is present in the tumour cells.

One embodiment of the present invention is a method for predicting the response to a topoisomerase IIα inhibitor therapy in an individual having cancer, said method comprising the steps of:
a. determining in a sample obtained from said individual, the presence of a TIMP-1 DNA aberration in the tumour cells of said sample, and
b. determining the presence of any TOP2A DNA aberration in the tumour cells of said sample
c. classifying the individual as having a high likelihood of responding to a topoisomerase IIα inhibitor therapy if a TOP2A DNA aberration is present and/or if said tumour cells comprise said TIMP-1 DNA aberration on either or both of the alleles of the TIMP-1 gene, and
d. classifying the individual as having a low likelihood of responding to a topoisomerase IIα inhibitor therapy if no TOP2A DNA aberration is present and if neither of the TIMP-1 alleles comprise said TIMP-1 DNA aberration.

Another embodiment of the present invention is a method for predicting the response to a topoisomerase IIα inhibitor therapy in an individual having cancer, said method comprising the steps of:
a. determining in a sample obtained from said individual, the presence of a TIMP-1 DNA aberration in the tumour cells of said sample, and
b. determining the presence of any HER2 DNA aberration in the tumour cells of said sample
c. classifying the individual as having a high likelihood of responding to a topoisomerase IIα inhibitor therapy if a HER2 DNA aberration is present and/or if said tumour cells comprise said TIMP-1 DNA aberration on either or both of the alleles of the TIMP-1 gene, and
d. classifying the individual as having a low likelihood of responding to a topoisomerase IIα inhibitor therapy if no HER2 DNA aberration is present and if neither of the TIMP-1 alleles comprise said TIMP-1 DNA aberration.

The TOP2A and HER2 genes are both located in the TOP2A/HER2 amplicon on chromosome 17q21 while the TIMP-1 gene is located on chromosome X.

The methods provide a means of identifying, without reducing the hazard ratio, almost twice the number of cancer patients compared to conventional methods who have a high likelihood of benefiting from an anti-cancer therapy such as CEF treatment.

In one embodiment according to the invention, the sample comprising the biomarkers (HER2, TOP2A and TIMP-1) is selected from the group consisting of a tumour tissue sample, a blood sample, a plasma sample, a serum sample, a urine sample, a faeces sample, a saliva sample, and a sample of serous liquid from the thoracic or abdominal cavity and a combination hereof.

One embodiment of the invention relates to a method for predicting the response to an anti-cancer therapy in an individual having a cancer selected from the group consisting of breast cancer, sarcomas, ovarian cancer and lung cancer.

In one embodiment the sarcomas may be soft tissue sarcomas.

In another embodiment the lung cancer may be non small cell lung cancer.

In one preferred embodiment the present invention pertains to a method for predicting the response to an anti-cancer therapy in an individual having a breast cancer.

### Methods of Measuring DNA Aberrations

Aberrations relating to DNA aberrations may be determined by means of DNA measurement such as but not limited to in situ hybridization, a PCR method, differential display, DNA-dot-blotting, Southern blotting or combinations hereof.

Thus in one embodiment, the level of DNA gene aberration is determined by means of DNA measurement such as but not limited to in situ hybridization, a PCR method, differential display, DNA-dot-blotting, Southern blotting or combinations hereof.

In a preferred embodiment, said in situ hybridization is determined by means of FISH (Fluorescent In-Situ Hybridization).

In yet a preferred embodiment, DNA aberrations are determined by FISH comprising the use of a probe mixture comprising labeled DNA probes targeted at a portion of the TOP2A gene region, and/or the HER2 gene region, and/or a portion of the TIMP-1 gene region and a probe mixture comprising fluoroscein-labelled probes targeted at the centromeric region of chromosome 17 and the X chromosome, respectively.

Aberrations relating to protein expression aberrations may be determined by means of Western blotting, Immunohistochemistry, ELISA, or RIA.

Thus in one embodiment, aberrant protein expression is determined by means of protein level measurement such as Western blotting, Immunohistochemistry, Immunocytology, ELISA, and RIA.

DNA aberrations and/or aberrant protein expression may also be reflected in the level of RNA such as mRNA transcripts of the gene in questions for example aberrant splicing of the primary transcript resulting in non-functional transcripts.

Thus a DNA aberration resulting in a RNA aberration may be determined by means of RNA such as mRNA measurement such as but not limited to Northern blotting, RNA dot and a quantitative PCR method.

Thus in one embodiment, the DNA aberration or a protein expression in the tumour cells correlate with aberrant mRNA levels in the tumour cells of said sample.

### DNA Aberrations

DNA aberrations refer to any DNA aberrations within a chromosome including specific regions of a chromosome such as an amplicon, and any DNA aberrations within a gene or region of a gene. DNA aberrations comprise DNA amplification, DNA deletion, gene point mutation, and translocation, epigenetic modifications of DNA such as DNA methylation, and combinations hereof. DNA aberrations comprise any DNA aberration resulting in downstream aberrant transcription of said DNA or protein expression of a protein encoded by said DNA. DNA aberrations in the meaning of deletion or amplification refer to deletion or amplification or entire gene or a part of said gene. Epigenetic aberrations may lead to silencing of the gene in question and is reflected in absence of the protein encoded by said gene or at least aberrant protein expression.

Thus, one embodiment relates to a method for predicting the response to a topoisomerase IIα inhibitor therapy in an individual having cancer, comprising the determination of TOP2A gene aberration, wherein said gene aberration is selected from the group consisting of TOP2A DNA amplification, TOP2A DNA deletion, TOP2A gene point mutation, and TOP2A DNA translocation, epigenetic modifications of the TOP2A DNA such as DNA methylation, and combinations hereof.

In a particular embodiment, the TOP2A DNA aberration or the increase in topoisomerase IIα protein in the tumour cells correlate with aberrant TOP2A mRNA levels in the tumour cells of said sample.

A further embodiment relates to a method for predicting the response to a topoisomerase IIα inhibitor therapy in an individual having cancer, comprising the determination of HER2 gene aberration, wherein the HER2 gene aberration is selected from the group consisting of HER2 gene amplification, HER2 DNA deletion, HER2 gene point mutations and HER2 DNA translocations, epigenetic modifications of the HER2 DNA such as DNA methylation, and combinations hereof.

In a particular embodiment, the HER2 DNA aberration or an increase in ErbB2 protein in the tumour cells correlate with aberrant HER2 mRNA levels in the tumour cells of said sample.

A further embodiment relates to a method for predicting the response to a topoisomerase IIα inhibitor therapy in an individual having cancer, comprising the determination of TIMP-1 gene aberration, wherein the tumour cells comprise at least one TIMP-1 DNA aberration resulting in lack of TIMP-1 protein expression selected from the list consisting of a deletion of one of the TIMP-1 alleles, a deletion of both of the TIMP-1 alleles, a partial deletion of one of the TIMP-1 alleles, a partial deletion of both of the TIMP-1 alleles, TIMP-1 DNA point mutations, TIMP-1 DNA inversion, TIMP-1 DNA translocation, epigenetic modifications of the TIMP-1 DNA such as DNA methylation, and combinations hereof.

In a particular embodiment, the any TIMP-1 DNA aberration or absence of TIMP-1 protein in the tumour cells correlate with aberrant TIMP-1 mRNA levels in the tumour cells of said sample such as absence of TIMP-1 mRNA in said sample.

In the present context the term “absence of TIMP-1 protein” is to be understood as total lack of TIMP-1 immunoreactivity in the cancer cells and/or the tumor tissue stromal cells. It should be stated however, that patients with weak TIMP-1 immunoreactivy in their cancer cells and/or the tumor tissue stromal cells have more benefit from anthracyclines than patients with stronger TIMP-1 immunoreactivity in their cancer cells and/or the tumor tissue stromal cells, while these patients with weak TIMP-1 immunoreactivity have less benefit from anthracycline treatment than patients with total absence of TIMP-1 immunoreactivity in their cancer cells and/or the tumor tissue stromal cells. Evaluation of TIMP-1 immunoreactivity (number of positive cells and/or intensity) can be evaluated by simple microscopy but can also be objectively estimated by a digitized analyser.

The cells are classified as 0, +1, +2 and +3. 0 is to be understood as the cancer cells and/or the tumor tissue stromal cells absent in TIMP-1 immunoreactivity, +1 is to be understood as the cancer cells and/or the tumor tissue stromal cells having week TIMP-1 immunoreactivity. +2 is to be understood as the cancer cells and/or the tumor tissue stromal cells having TIMP-1 immunoreactivity. +3 is to be understood as the cancer cells and/or the tumor tissue stromal cells having strong TIMP-1 immunoreactivity.

The method of classifying and differentiating TIMP-1 immunoreactivity is in an embodiment of the invention objectively evaluated. The evaluation is based on the number of TIMP-1 immunoreactive cells (cancer and/or tumor tissue stromal cells) and/or the intensity of the immunoreactivity. Evaluation of TIMP-1 immunoreactivity (number of positive cells and/or intensity) can be evaluated by simple microscopy but can also be objectively estimated by a digitized analyser.

Thus, in a preferred embodiment of the present invention cancer cells and/or tumor tissue stromal cells are absent in TIMP-1 if the immunoreactivity is below +1, such as below +0.9, e.g. below +0.8, such as below +0.7, e.g. below 0.6, such as below 0.5, e.g. below 0.4, such as below 0.3, e.g. below 0.2, such as below 0.1.

Thus, in a preferred embodiment of the present invention cancer cells and/or tumor tissue stromal cells are absent in TIMP-1 if the immunoreactivity is 0.

Thus, in a preferred embodiment of the present invention a patient is likely to benefit from anthracyclines (e.g. topoisomerase IIα) if the level of TIMP-1 immunoreactivity is below +2, such as below +1.9, e.g. below +1.8, such as below +1.7, e.g. below 1.6, such as below 1.5, e.g. below 1.4, such as below 1.3, e.g. below 1.2, such as below +1, such as below +0.9, e.g. below +0.8, such as below +0.7, e.g. below 0.6, such as below 0.5, e.g. below 0.4, such as below 0.3, e.g. below 0.2, such as below 0.1. Preferably in the range from 0-+2, e.g. in the range from 0.1-+1,5, such as in the range from +0.5-+1.2, e.g. in the range from 0-+0.5, such as in the range from 0-+1.

In a preferred embodiment a patient is likely to benefit from anthracyclines (e.g. topoisomerase IIα inhibitor) if the level of TIMP-1 immunoreactivity is below +1, such as below +0.9, e.g. below +0.8, such as below +0.7, e.g. below 0.6, such as below 0.5, e.g. below 0.4, such as below 0.3, e.g. below 0.2, such as below 0.1.

In a preferred embodiment a patient is likely to benefit from anthracyclines (e.g. topoisomerase IIα inhibitor) if the level of TIMP-1 protein is 0.

It is to be understood that TIMP-1 immunoreactivity resembles the amount of TIMP-1 protein present in the cancer cell and/or the tumor tissue stromal cell.

In another embodiment the TIMP-1 gene is more than 1.1 fold amplified relative to a reference sample, such as more than 1.2 fold, e.g. more than 1.3 fold, such more than 1.4 fold, e.g. more than 1.5 fold, such as more than 1.6 fold, e.g. more than 1.7 fold, such as more than 1.8 fold, e.g. more than 1.9 fold, such as more than, such as more than 3 fold, for example more than 4 fold, such as more than 5 fold, for example more than 6 fold, such as more than 7 fold, for example more than 8 fold, such as more than 9 fold, for example more than 10 fold, such as more than 15 fold, for example more than 20 fold, such as more than 30 fold, for example more than 40 fold, such as more than 50 fold, for example more than 100 fold of a reference sample. In an embodiment of the present invention the TIMP-1 gene is between 1.1-2.0 amplified relative to a reference sample, such as in the range from 1.2-1.9, e.g. in the range from 1.3-1.8, such as in the range from 1.4-1.7, e.g. in the range from 1.5-1.7, such as in the range from 1.7-1.9, e.g. in the range from 1.8-1.9 amplified relative to a reference sample.

In another embodiment the TOP2A gene is more than 1.1 fold amplified relative to a reference sample, such as more than 1.2 fold, e.g. more than 1.3 fold, such more than 1.4 fold, e.g. more than 1.5 fold, such as more than 1.6 fold, e.g. more than 1.7 fold, such as more than 1.8 fold, e.g. more than 1.9 fold, such as more than, such as more than 3 fold, for example more than 4 fold, such as more than 5 fold, for example more than 6 fold, such as more than 7 fold, for example more than 8 fold, such as more than 9 fold, for example more than 10 fold, such as more than 15 fold, for example more than 20 fold, such as more than 30 fold, for example more than 40 fold, such as more than 50 fold, for example more than 100 fold of a reference sample. In an embodiment of the present invention the TOP2A gene is between 1.1-2.0 amplified relative to a reference sample, such as in the range from 1.2-1.9, e.g. in the range from 1.3-1.8, such as in the range from 1.4-1.7, e.g. in the range from 1.5-1.7, such as in the range from 1.7-1.9, e.g. in the range from 1.8-1.9 amplified relative to a reference sample.

In another embodiment the HER2 gene is more than 1.1 fold amplified relative to a reference sample, such as more than 1.2 fold, e.g. more than 1.3 fold, such more than 1.4 fold, e.g. more than 1.5 fold, such as more than 1.6 fold, e.g. more than 1.7 fold, such as more than 1.8 fold, e.g. more than 1.9 fold, such as more than, such as more than 3 fold, for example more than 4 fold, such as more than 5 fold, for example more than 6 fold, such as more than 7 fold, for example more than 8 fold, such as more than 9 fold, for example more than 10 fold, such as more than 15 fold, for example more than 20 fold, such as more than 30 fold, for example more than 40 fold, such as more than 50 fold, for example more than 100 fold of a reference sample. In an embodiment of the present invention the HER2 gene is between 1.1-2.0 amplified relative to a reference sample, such as in the range from 1.2-1.9, e.g. in the range from 1.3-1.8, such as in the range from 1.4-1.7, e.g. in the range from 1.5-1.7, such as in the range from 1.7-1.9, e.g. in the range from 1.8-1.9 amplified relative to a reference sample.

### Aberrant Protein Expression

Aberrant protein expression refers to any aberration in the protein expression such as the level of said protein, absence of said protein, dysfunctions in terms of functionality for example a mutation causing a non-functional protein, dysfunctions in terms of cellular localisation of said protein.

Absence usually refers to the absence of detectable protein in a sample or in tumour cells of said sample.

In one embodiment, the aberrant protein expression is determined as fold over a reference level of a control sample. In another embodiment, the aberrant protein expression is determined as fold under a reference level.

In a second embodiment relating to aberrant topoisomerase IIα protein expression, topoisomerase IIα protein is more than 2 fold over-expressed relative to a reference sample, such as more than 3 fold, for example more than 4 fold, such as more than 5 fold, for example more than 6 fold, such as more than 7 fold, for example more than 8 fold, such as more than 9 fold, for example more than 10 fold, such as more than 15 fold, for example more than 20 fold, such as more than 30 fold, for example more than 40 fold, such as more than 50 fold, for example more than 100 fold of a reference sample.

In a second embodiment relating to aberrant ErbB2 protein expression, ErbB2 protein is more than 2 fold over-expressed relative to a control sample, such as more than 3 fold, for example more than 4 fold, such as more than 5 fold, for example more than 6 fold, such as more than 7 fold, for example more than 8 fold, such as more than 9 fold, for example more than 10 fold, such as more than 15 fold, for example more than 20 fold, such as more than 30 fold, for example more than 40 fold, such as more than 50 fold, for example more than 100 fold of a control sample.

In a preferred embodiment of the present invention is a method for predicting the response to a topoisomerase IIα inhibitor therapy in an individual having cancer, wherein the tumour cells are absent of TIMP-1 protein.

### Reference

The “reference” refers to any suitable reference such as corresponding measurements on a pool of corresponding biological sample from a non-cancer individual or to non-malignant cells in a tumor, e.g. tumor tissue stromal cells.

A method for predicting the response to a topoisomerase IIα inhibitor therapy in an individual having cancer, wherein a reference obtained from a population is used to determine the level of DNA aberration or protein expression.

Said reference may be used to set the baseline of a signal such as TIMP-1, ErbB2, or topoisomerase IIα immunoreactivy in a sample in order to determine whether TIMP-1, ErbB2, or topoisomerase IIα protein is aberrantly expressed in a sample such as a sample applied to an ELISA assay.

In a particular embodiment, the reference is used to set a baseline/cut-off value for determining the presence or absence of TIMP-1 protein in a sample such as determining the presence or absence of TIMP-1 protein by means of Western blotting, Immunohistochemistry, ELISA, flow cytometry, or RIA.

In one embodiment the reference is selected from the group consisting of intra-sample, inter-sample and internal reference.

One example of a method according to the invention comprising the determination of DNA aberrations of a gene in question, wherein a reference is included targeting to the same chromosome. Thus for a DNA aberration in the TOP2A/HER2 amplicon on chromosome 17q21, such a DNA aberration in the TOP2A gene or HER2 gene, a reference targeting the centromeric of region of chromosome 17 may be used to determine whether an allele of the gene in question has been deleted or amplified.

Accordingly one embodiment concerns a method for predicting the response to a topoisomerase IIα inhibitor therapy according to the invention, wherein a DNA aberration is determined by means of in situ hybridization such as FISH (Fluorescent In-Situ Hybridization).

In another embodiment, said DNA aberration is determined as the average ratio to an internal reference sequence comprised in said sample. In one embodiment said internal reference is diploid non-malignant cells comprised in the samples for a tumor tissue sample. In a preferred embodiment of the present invention the tumour tissue sample is tumor tissue stromal cells.

In a further embodiment, the reference is the signal of a labelled probe such as a fluoroscein-labelled or Texas Red-5-labelled targeted at the centromeric region of chromosome 17 and/or the X chromosome. In a particular embodiment, the probe is a peptide nucleotide acid (PNA) based probe. This type of reference is suitable for FISH applications such as a FISH assays for determining a DNA aberration in TOP2A/HER2 amplicon on chromosome 17q21, for example a DNA aberration in the TOP2A gene or HER2 gene. In another embodiment, a similar type of reference is used in FISH assays for determining a DNA aberration in the TIMP-1 gene.

The DNA aberration may be determined as the average ratio to a reference sequence comprised in said sample.

Thus, in one embodiment the DNA aberration is determined as the average ratio to an internal reference sequence comprised in said sample.

In one embodiment, the internal reference sequence is located on the centromeric region of chromosome 17.

In a particular embodiment, the internal reference sequence is chromosome X α-satellite (Cen X).

DNA aberrations such as DNA gene allele deletions or gene amplifications may be determined using ratios of the signal corresponding to binding of the gene specific probe versus the signal corresponding to binding of centromeric region probe of the reference probe.

Accordingly, in one embodiment, the tumour cells of the sample comprise a TIMP-1 gene deletion if the average ratio of TIMP-1/Cen X is below 0.8, and normal if the said ratio is above 0.8 and below 2.0. In an embodiment of the present invention the average ratio of TIMP-1/Cen X is below 0.7, e.g. below 0.6, such as below 0.5, e.g. below 0.4, such as below 0.3, e.g. below 0.2, such as below 0.1, e.g. in the range from 0.1-0.8, such as in the range from 0.2-0.7, e.g. in the range from 0.3-0.6, such as in the range from 0.4-0.5 and normal if the said ratio is above 0.8 and below 2.0.

In another embodiment, the tumour cells comprise TOP2A gene deletion if the average ratio of TOP2A/Cen X is below 0.8 or amplifications if the average ratio of TOP2A/Cen X is above 2.0, and normal if the said ratio is above 0.8 and below 2.0. In an embodiment of the present invention the average ratio of TOP2A/Cen X is below 0.7, e.g. below 0.6, such as below 0.5, e.g. below 0.4, such as below 0.3, e.g. below 0.2, such as below 0.1, e.g. in the range from 0.1-0.8, such as in the range from 0.2-0.7, e.g. in the range from 0.3-0.6, such as in the range from 0.4-0.5 and normal if the said ratio is above 0.8 and below 2.0.

In third embodiment, the tumour cells comprise HER2 gene deletion average ratio of TOP2A/Cen X is below 0.8 or amplifications if the average ratio of HER2/Cen X is above 2.0, and normal if the said ratio is above 0.8 and below 2.0. In an embodiment of the present invention the average ratio of HER2/Cen X is below 0.7, e.g. below 0.6, such as below 0.5, e.g. below 0.4, such as below 0.3, e.g. below 0.2, such as below 0.1, e.g. in the range from 0.1-0.8, such as in the range from 0.2-0.7, e.g. in the range from 0.3-0.6, such as in the range from 0.4-0.5 and normal if the said ratio is above 0.8 and below 2.0.

In another embodiment, a reference is used to determine the level of DNA aberration or protein expression. The said reference may be obtained from a population such as a population of non-cancer individuals, or a combined group of cancer individuals for example a group of CMF treated cancer individuals.

In yet another embodiment, said reference is a normal diploid genetic background.

For example, a suitable reference for determining the TOP2A DNA aberration level in the meaning TOP2A DNA gene amplifications, or TOP2A DNA gene deletions, is the average signal from TOP2A DNA alleles in a corresponding biological sample from a non-cancer individual or the average signal in the non-malignant cells in said tumor sample.

In one embodiment, the determination of DNA or protein aberrations is performed on archive material from the individual, such as a paraffin block comprising tumour tissue.

### The Topoisomerase IIα Inhibitor Therapy

In one embodiment, the topoisomerase IIα inhibitor therapy comprises the administration of a composition comprising a least one topoisomerase IIα inhibitor to the individual with a cancer. In a preferred embodiment the composition used for the topoisomerase IIα inhibitor therapy comprises at least one anthracycline selected from the group consisting of 4-Epirubricin, Daunorubicin, Daunorubicin (liposomal), Doxorubicin, Doxorubicin (liposomal), Epirubicin, Idarubicin, and Mitoxantrone.

The topoisomerase IIα inhibitor may be administrated either alone or in combination with at least one other chemotherapeutic. In one embodiment according to the invention the topoisomerase IIα inhibitor therapy is CEF treatment, wherein CEF refers to Cyclophosfamide, 4-Epirubricin and 5-Fluorouracil. In yet another embodiment topoisomerase IIα inhibitor therapy is treatment with cyclophosphamide, taxanes and/or 5-fluorouracil in addition to a topoisomerase IIα inhibitor.

Any of the compounds used in the topoisomerase IIα inhibitor therapy may be administered as a prodrug. Thus, in one embodiment at least one of the drugs selected from the group consisting of cyclophosphamide, taxanes, 5-fluorouracil topoisomerase IIα inhibitor such as an anthracycline is in the form of a prodrug of said drug.

The topoisomerase IIα inhibitor therapy may be liposome encapsulated.

In one embodiment the topoisomerase IIα inhibitor therapy comprises an inducer of apoptosis or mitotic catastrophe.

In another embodiment the topoisomerase IIα inhibitor therapy is selected from the group consisting of neoadjuvant therapy, adjuvant therapy and therapy of metastatic disease.

### Method of Treating Cancer

Another aspect of the invention relates to the treatment of cancer based on the prediction of the likelihood of responding to a topoisomerase IIα inhibitor therapy.

Said aspect concerns a method of treating cancer in an individual comprising
a. predicting the response to a topoisomerase IIα inhibitor therapy according to any of the preceeding claims,
b. selecting a topoisomerase IIα inhibitor therapy to which said individual has a high likelihood of responding to, and
c. subjecting to said individual to said topoisomerase IIα inhibitor therapy.

one embodiment of said method of treatment, the topoisomerase IIα inhibitor is a anthracyclines selected from the group consisting of but not limited to 4-Epirubricin, Daunorubicin, Daunorubicin (liposomal), Doxorubicin, Doxorubicin (liposomal), Epirubicin, Idarubicin, and Mitoxantrone, or a combination hereof.

In a further embodiment the topoisomerase IIα inhibitor therapy is comprised in a composition further comprising cyclophosphamide and 5-fluorouracil.

In a further embodiment the topoisomerase IIα inhibitor therapy is comprised in a composition further comprising a taxane.

### Kit

A third aspect of the present invention relates to a kit for predicting the response to a topoisomerase IIα inhibitor therapy comprising:
a. reagents suitable for the determination of a chromosomal DNA aberration in the TOP2A/HER2 amplicon such as TOP2A or HER2 DNA aberrations in a biological sample, and
b. reagents suitable for the determination of a TIMP-1 DNA aberration or determining the level of a TIMP-1 protein in a biological sample.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### Hazard Ratio

“Hazard ratio” (HR) refers to likelihood of obtaining benefit such as prolonged disease free survival from a treatment such as a topoisomerase IIα inhibitor therapy.

In one embodiment of the present invention HR describes the likelihood of having benefit from CEF treatment with the benefit from CMF treatment as the reference. A HR of 1 means no difference between the group receiving the treatment and the reference group. Accordingly, a HR of 0.5 means that the CEF treated patients have 50% reduced risk of experiencing a relapse as compared to CMF treated patients. Confidence intervals may be included to improve the statistic power of the evaluation.

Table 1 of Example 1 exemplifies the use of hazard ratios in order to evaluate likelihood of obtaining benefit from a treatment such as a topoisomerase IIa inhibitor therapy. The HR of the reference group (in this case CMF treated patients) is set to 1.

Accordingly, a preferred embodiment of the present inventions relates to a method for predicting the response to a topoisomerase IIα inhibitor therapy in an individual having cancer, wherein the likelihood of responding to a topoisomerase IIα inhibitor therapy is determined by means of a hazard ratio.

### DEFINITIONS

Prior to discussing the present invention in further details, the following terms and conventions will first be defined:

“Anti-cancer therapy” is a term used for any non-surgical therapeutic regimen that aims at curving or alleviating cancer. Examples are set forth below but anti-cancer therapy can be both chemotherapeutic and/or radiotherapeutic and/or anti-hormonal and/or biological therapy.
“A topoisomerase IIα inhibitor therapy” refers to chemotherapeutic anti-cancer therapy comprising the use of at least one topoisomerase IIα inhibitor. A topoisomerase IIα inhibitor may be administrated in combination with other chemotherapeutic drugs such as cyclophosphamide, taxanes and/or 5-fluorouracil.
“Anthracycline” refers to a group of topoisomerase IIα inhibitors 4-Epirubricin, Daunorubicin, Daunorubicin (liposomal), Doxorubicin, Doxorubicin (liposomal), Epirubicin, Idarubicin, and Mitoxantrone.

The present invention will hereinafter be described by way of the following non-limiting Figures and Examples.

### FIGURE LEGENDS

FIG. 1A shows a Kaplan Meier plot illustrating disease free survival of patients receiving adjuvant CEF. The patients were stratified according to tumor cell TIMP-1 immunoreactivity scored as + or − immunoreactivity in the cancer cells. The number of patients at risk at selected time points is given below the x-axis.

FIG. 1B shows a Kaplan Meier plot illustrating disease free survival of patients receiving adjuvant CMF. The patients were stratified according to tumor cell TIMP-1 immunoreactivity scored as + or − immunoreactivity in the cancer cells. The number of patients at risk at selected time points is given below the x-axis

FIG. 1C shows the Kaplan Meier curves which show the disease free survival of patients without TIMP-1 immunoreactivity in their cancer cells treated with CEF or CMF.

FIG. 2A shows a Kaplan Meier plot illustrating disease free survival of patients receiving adjuvant CEF. The patients were stratified according to the presence or absence of tumor cell TOP2A DNA aberrations. The number of patients at risk at selected time points is given below the x-axis.

FIG. 2B shows a Kaplan Meier plot illustrating disease free survival of patients receiving adjuvant CMF. The patients were stratified according to the presence or absence of tumor cell TOP2A DNA aberrations. The number of patients at risk at selected time points is given below the x-axis

FIG. 2C shows the Kaplan Meier curves which show the disease free survival of patients with TOP2A DNA aberrations in their cancer cells treated with either CEF or CMF.

FIG. 3A shows a Kaplan Meier plot illustrating disease free survival of patients receiving adjuvant CEF. The patients were stratified according to tumor cell TIMP-1 immunoreactivity scored as + or − immunoreactivity in the cancer cells and presence (Ab) or absence (normal) of TOP2A DNA aberrations. The number of patients at risk at selected time points is given below the x-axis.

FIG. 3B shows a Kaplan Meier plot illustrating disease free survival of patients receiving adjuvant CMF. The patients were stratified according to tumor cell TIMP-1 immunoreactivity scored as + or − immunoreactivity in the cancer cells and the presence (Ab) or absence (normal) of TOP2A DNA aberrations. The number of patients at risk at selected time points is given below the x-axis

FIG. 3C shows the Kaplan Meier curves which show the disease free survival of patients without TIMP-1 immunoreactivity and/or with TOP2A DNA aberrations in their cancer cells treated with CEF or CMF.

FIGS. 4A and 4B Kaplan-Meier curves for invasive disease-free survival by treatment with CMF or CEF and HT (HER2 and TIMP-1) status (Panel 4A) and 2T (TOP2A and TIMP-1) status (Panel 4B).

FIGS. 5A and 5B Forest plots illustrating hazard ratio estimates of treatment effect for invasive disease-free survival (Panel 5A) and overall survival (Panel 5B) comparison between patients with HER2 positive and HER2 negative tumors, TOP2A DNA aberrant and non-aberrant (normal) tumors, TIMP-1 positive and negative tumors, HT responsive and non-responsive tumors and 2T responsive and non-responsive tumors.

FIG. 6A-D This Figure shows examples of TIMP-1 immunohistochemistry. 6A: A large proportion of the epithelial cancer cells are TIMP-1 positive. 6B: Scattered and focalized TIMP-1 immunoreactivity in the epithelial cancer cells. 6C: Negative control. 6D: TIMP-1 immunoreactivity in fibroblasts but not in the epithelial cancer cells.

FIGS. 7A and 7B Invasive Disease-Free Survival (IDFS) (FIG. 7A) and overall survival (OS) (FIG. 7B) probabilities for breast cancer patients with known TIMP-1 status. T+ and T− means patients with and without TIMP-1 immunoreactivity in their breast cancer cells, respectively. CEF and CMF refer to received adjuvant chemotherapy.

FIGS. 8A and 8B Forest plots illustrating hazard ratios from multivariate models for effect of CEF with CMF as baseline in TIMP-1 subgroups and ER subgroups of patients. FIG. 8A: IDFS; FIG. 8B: OS

FIG. 9 TIMP-1 FISH analysis showing TIMP-1 DNA amplifications in the epithelial breast cancer cells

### EXAMPLES

In the present context the following aberrations are used

### DBCG: Danish Breast Cancer Cooperative Group

### CMF: Cyclophosphamide, Methotrexate and 5-Fluorouracil

CEF: Cyclophosphamide, 4.epi-adriamycin and 5-Fluorouracil
CAF: Cyclophosphamide, 4.epi-adriamycin and 5-Fluorouracil
TOP2A normal: No DNA aberrations found in the TOP2A gene
HER2 normal: No DNA aberrations found in the HER2 gene
HT-sensitive: HER2 gene amplification or 3 plus for Her2 immunohistochemistry and TIMP-1 negative
2T-sensitive: TOP2A gene aberrations and TIMP-1 negative

### TMA: Tissue Micro Arrays

ER or ER immunostaining: Immunostaining for estrogen or progesterone receptors
FISH: Fluorescence in situ hybridization

### IHC: Immunohistochemistry

### IDFS: Invasive Disease Free Survival

OS: Overall survival

### Example 1

Lack of TIMP-1 tumour cell immunoreactivity predicts effect of adjuvant anthracycline based chemotherapy in patients (n=647) with primary breast cancer.

### Methods

### Patients and Methods

Briefly, DBCG (Danish Breast Cancer Cooperative Group) trial 89D was an open-labeled randomized, phase III trial comparing CEF (Cyclophosphamide, Epirubicin and Fluorouracil) against CMF (Cyclophosphamide, Methotrexate and Fluorouracil). Eligible for the 89D trial were patients with node positive (or tumor size≧5 cm) and hormone receptor negative breast cancer, and premenopausal patients with node negative and malignancy grade II or III tumours. All patients gave informed consent to the trial. The DBCG 89D trial did not include patients with node positive, hormone receptor positive tumours. These patients were included in trials with endocrine treatment. The DBCG prepared the original protocol as well as the biomarker supplements and The Danish National Committee on Biomedical Research Ethics approved the original protocol as well as the supplements before their activation.

### Pathology Assessments

The pathological procedure included classification of histological type according to WHO, examination of tumour margins, invasion into skin or deep fascia, measurement of gross tumour size, number of metastatic and total number of lymph nodes identified. All invasive ductal carcinomas were graded for malignancy. All sections have subsequently been analysed centrally for ER by immunohistochemistry and these centrally obtained ER data were used in the present analyses. Tumours with 10% stained tumour cells were considered ER positive.

Retrospective collection of archival tumour tissue and construction of TMA's From June 1990 to January 1998, 1224 patients were randomized in the DBCG trial 89D and 980 of these were recruited in Denmark. Archival paraffin embedded tissue blocks from 806 Danish patients enrolled in the trial were collected between September 2001 and August 2002 from the study sites and stored centrally. Tissue Micro Arrays (TMA) were successfully constructed from 707 of 797 blocks still assessable by means of a TMA-builder from Histopathology Ltd (AH-diagnostics, Denmark). A target area was identified in the donor block on haematoxylin stained sections and two 2 mm tissue cores were transferred to the recipient TMA block. For orientation the upper corners were marked using cores of kidney tissue. For the present study, a total number of 659 tumours were available for TIMP-1 analysis. The lack of tumours (659-707) was due to their prior use in other studies resulting in no left-over tissue for the present study. Table 7 shows the flow of the patients in the study

### TIMP-1 Immunostaining

The mouse monoclonal antibody (clone VT7) raised against recombinant human TIMP-1 was included. The present inventions have previously validated this antibody for immunostaining. The VT7 antibody is of the IgG₁ subtype and was used in the concentration 0.25 μg/ml. In addition, an irrelevant IgG₁ monoclonal antibody (anti-TNP) raised against tri-nitro-phenol hapten was used as control. For each immunohistochemical experiment, a positive control case (human mammary carcinoma known to contain TIMP-1) was included. Reagents used for IHC staining were obtained from Dako A/S and were used according to the manufacturer's instructions.

In brief, paraffin sections (4 μm) were dewaxed in xylene and rehydrated through a graded series of ethanol. Antigen retrieval was carried out by boiling the sections for 10 minutes in a conventional microwave oven in 10 mM citrate buffer pH 6.00 followed by 30 minutes in the hot buffer at room temperature. To block endogenous peroxidase activity, the sections were treated with 1% hydrogen peroxide for 10 minutes. Sections were incubated with primary antibody overnight at 4° C. The monoclonal antibodies were detected with Advance HRP (Code no K4068), and the reactions were visualized by incubating the sections with DAB+ (Code No K5007) for 5 minutes. Washes between incubations were carried out with TBS containing 0.5% Triton x-100, pH 7.6. The sections were counterstained with Mayer's haematoxylin, and all staining procedures were performed manually.

Immunostaining of tissue sections was assessed semi-quantitatively using + and − symbols as a measure of TIMP-1 immunoreactivity in the epithelial breast cancer cells. Scoring of the intensity of the signal was not included. The scoring of the tissue sections was performed blinded by two independent pathologists (GW and EB). In case of discrepancies, agreement was reached by looking at the slides together.

### Statistical Methods

The immunostaining results were transferred to the DBCG secretariat for statistical analyses.

Follow-up time was quantified in terms of a Kaplan-Meier estimate of potential follow-up. IDFS (Invasive Disease-Free Survival) was the primary and OS (Overall Survival) the secondary end-point. IDFS was defined as the elapsed time from randomization until invasive breast cancer recurrence irrespective of localization, second primary invasive cancer or death attributable to any cause. OS was defined as the elapsed time from randomization until death attributable to any cause. IDFS and OS were analysed using Kaplan-Meier estimates and the log rank test. The effect of treatment regimen as well as centrally assessed TIMP-1 on IDFS and OS was quantified in terms of the hazard ratio, estimated unadjusted and adjusted using the Cox proportional hazards model. The multivariate Cox proportional hazards model was also applied to investigate interaction of treatment and TIMP-1 using the Wald test. The multivariate model included TIMP-1, menopausal status, tumour size, positive lymph nodes, histological type and grade, central ER hormone receptor status, treatment regimen and interaction terms of TIMP-1 and treatment. The proportional hazard assumptions were not fulfilled for histological type & grade and ER receptor status, and these were included in the model as stratification variables. Differences between patients with and without information about biomarkers, between treatment regimens, and correlations between TIMP-1 status and clinico-pathological variables were tested by χ²-test excluding unknowns. P-values are two-tailed. Statistical analyses were done with the SAS 9·1 program package.

### Results

The total number of tumour samples investigated was 659, among whom 12 did not receive CMF or CEF, resulting in a final number of 647 patients for subsequent analyses. 357 of these patients received CMF and 290 patients received CEF. Table 7 shows the flow of the original patients enrolled in the Danish part of DBCG 89D study and how we ended up with a total of 647 patients to be included in the final analysis. At the time of the present analyses (Aug. 1, 2007), 308 (48%) have died and 312 (48%) have had an event corresponding to IDFS. For the patients receiving CEF 123 (42%) had died and 129 (44%) had had an event corresponding to IDFS. Among CMF treated patients, 185 (52%) had died and 183 (51%) had had an IDFS event. The median potential follow-up time with respect to IDFS was 9.8 years and 13.8 years with respect to OS.

Table 5 shows the base-line characteristics of the intention to treat population. As can be seen, patients included in the present study had significantly larger tumours (p<0.0001) and significantly higher grade of malignancy (p=0.02) than the remaining patients. No significant differences were found for the other classical base-line characteristics. When dividing the 647 patients into the two treatment groups (CMF vs. CEF) no differences in base-line characteristics were observed, indicating that although approximately one third of the patients were lost for the present study, the included patients had retained a balanced distribution.

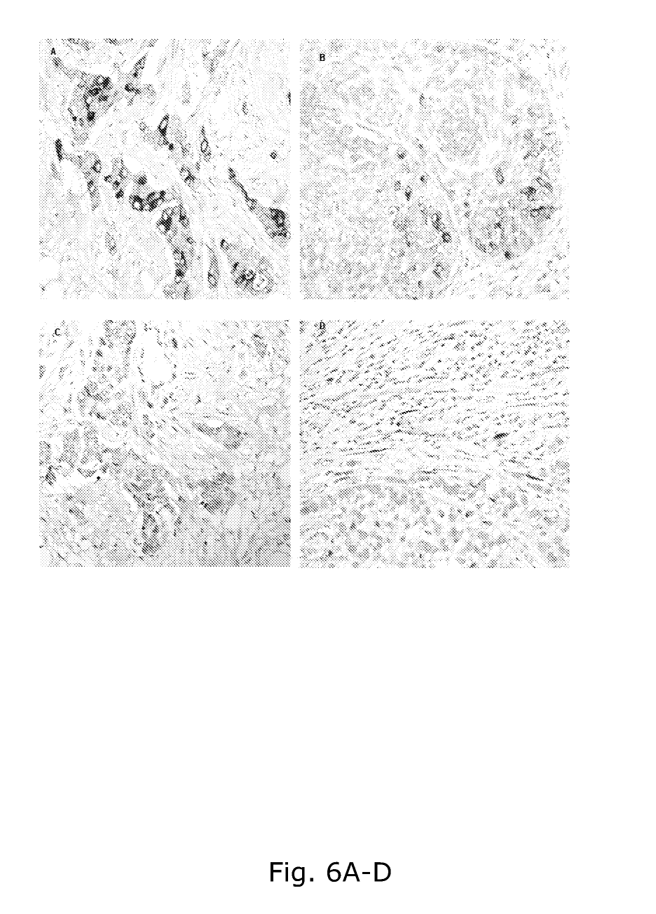

75% of the tumour samples showed positive TIMP-1 immunoreactivity. The pattern of immunoreactivity ranged from almost all epithelial cancer cells displaying TIMP-1 immunoreactivity (FIG. 6A) through scattered and focalized TIMP-1 immunoreactivity (FIG. 6B) (TIMP-1 positive) to total absence of TIMP-1 tumour cell immunoreactivity (not shown). In some tumours, distinct tumor tissue stromal cell TIMP-1 immunoreactivity was observed, but if these tumours were devoid of epithelial cancer cell TIMP-immunoreactivity, they were counted as TIMP-1 negatives (FIG. 6D). FIG. 6C is a negative control. Table 6 shows the base-line characteristics between patients having TIMP-1 positive and patients having TIMP-1 negative tumour cells. Patients with TIMP-1 positive tumour cells had significantly more tumour positive axillary lymph nodes (p=0.02) and significantly more ER positive tumours (p=0.04). Among the TIMP-1 negative tumors (n=160), the majority were ER-negative (n=107). However, among the TIMP-1 positive tumors (n=487) there was also a large proportion being ER-negative (n=294). This shows that even though TIMP-1 negativity primarily is found among ER-negative tumors, TIMP-1 is not a general surrogate for ER. No other differences in base-line characteristics between TIMP-1 negative/positive patients could be demonstrated.

The multivariate analysis (adjusted) included treatment arm, menopausal status, tumour size, number of positive axillary lymph nodes, histological type and malignancy grading, ER centrally measured and TIMP-1 tumour cell immunoreactivity. As stated above, the proportional hazard assumptions were not fulfilled for histological type & grade and ER receptor status, and these were therefore included in the multivariate model as stratification variables. The present inventors first analysed the effect on IDFS and OS of CEF versus CMF in the 647 patients included in the present study. Thus, TIMP-1 immunoreactivity in the cancer cells was not taken into consideration. Patients who received CEF had a superior IDFS (adjusted HR: 0.78 (95% CI: 0.62-0.98; p=0.03) and superior OS (adjusted HR=0.77 (95% CI: 0.61-0.97; p=0.03) when compared with patients receiving CMF (not shown). These figures are not different from those of the original study (IDFS: HR=0.76 and OS: HR=0.73) (Ejlertsen et al. 2007), suggesting that the studied subgroup is representative of the whole study group.

The present inventors then analysed the association between TIMP-1 cancer cell immunoreactivity and IDFS and OS for the whole included patient cohort (n=647). No significant differences were seen between TIMP-1 positive versus TIMP-1 negative patients with regard to IDFS; unadjusted HR=1.18 (95% CI: 0.91-1.54; p=0.22) and adjusted HR=0.95 (95% CI: 0.72-1.24; p=0.69). For OS the figures were: unadjusted HR=1.17 (95% CI: 0.89-1.53; p=0.25) and adjusted HR=0.97 (95% CI: 0.73-1.28; p=0.82).

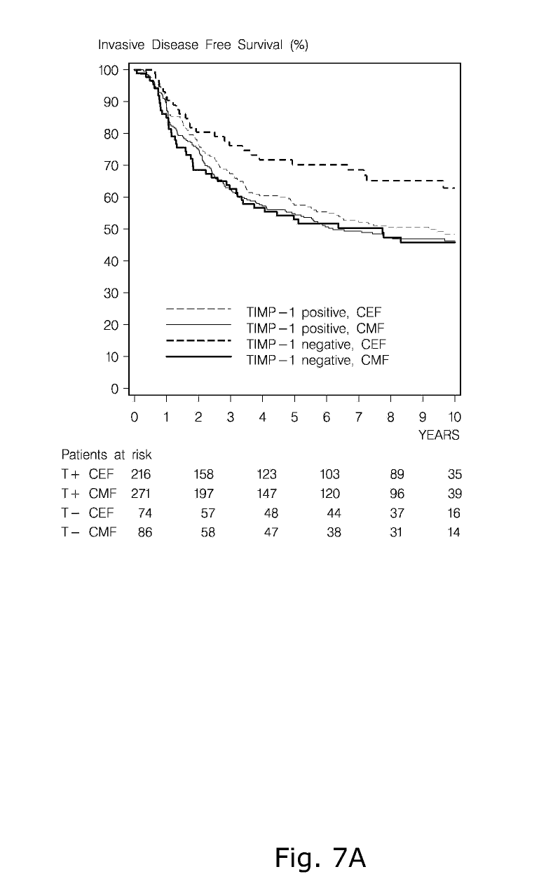

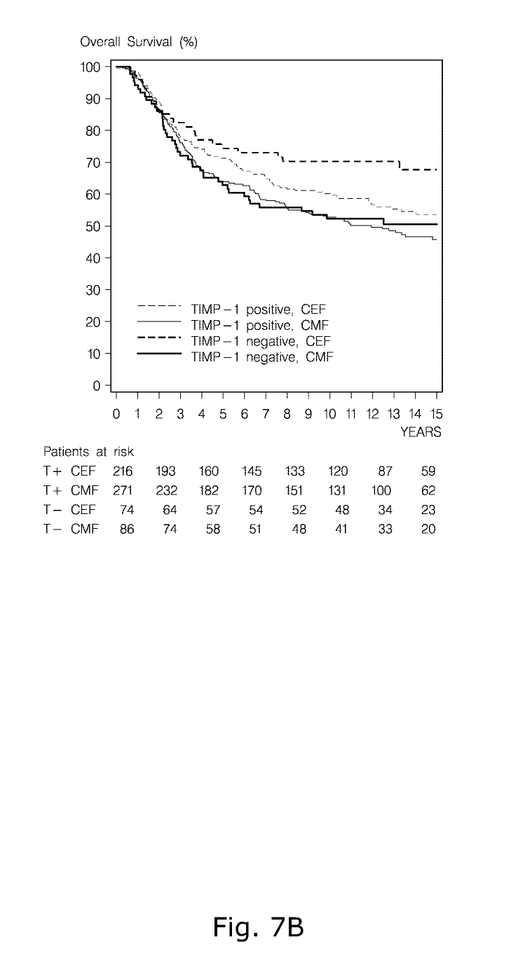

Subgroup analyses, taking the two different treatment arms and tumour cell TIMP-1 immunoreactivity into consideration, were then performed. In the CEF treated patients (n=290), individuals with TIMP-1 positive tumours had a significant shorter IDFS than patients with TIMP-1 negative tumours; unadjusted HR=1.56 (95% CI: 1.01-2.41; p=0.047) (FIG. 7A). In contrast, in the CMF treated patients (n=347), no differences in IDFS were seen between TIMP-1 positive and negative patients; unadjusted HR=0.97 (95% CI: 0.69-1.35; p=0.84) (FIG. 7A). The corresponding figures for OS were: CEF: unadjusted HR=1.41 (95% CI: 0.91-2.18; p=0.13) and CMF: unadjusted HR=1.02 (95% CI: 0.72-1.43; p=0.93) (FIG. 7B).

In the multivariate analyses, no significant differences were seen between TIMP-1 positive versus TIMP-1 negative patients treated with CEF with regard to IDFS: adjusted HR=1.30 (95% CI: 0.83-2.02; p=0.25) and OS: adjusted HR=1.21 (95% CI: 0.77-1.90; p=0.42. Nor were significant differences observed in patients treated with CMF; IDFS: adjusted HR=0.76 (95% CI: 0.54-1.07; p=0.12) or OS: adjusted HR=0.84 (95% CI: 0.59-1.19; p=0.32).

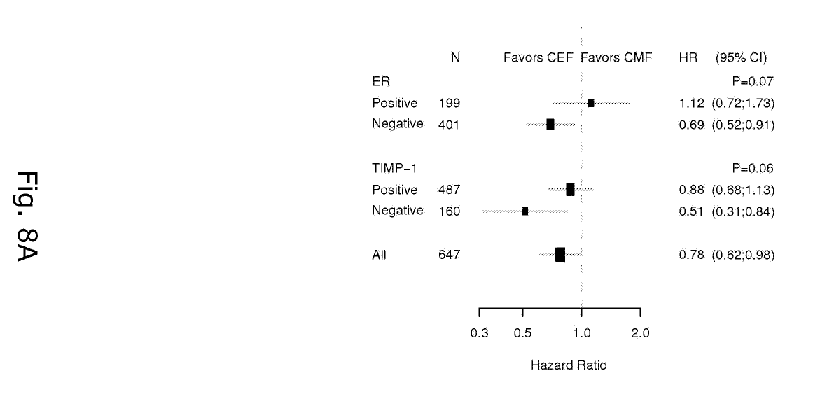

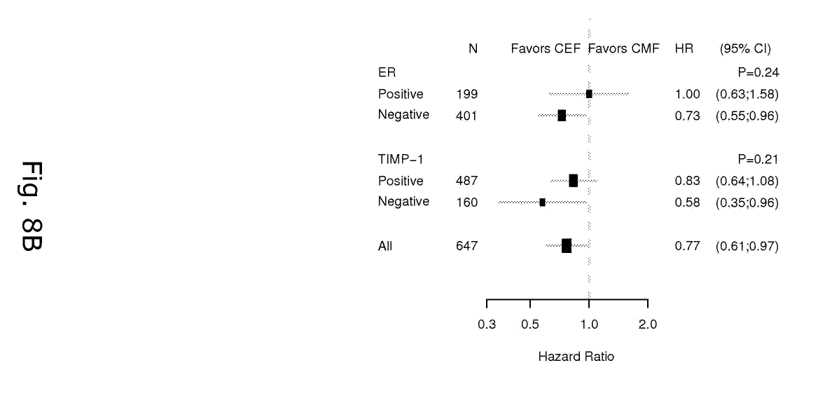

When comparing IDFS in CEF versus CMF treated patients in the group with TIMP-1 immunoreactive cancer cells the HR between the two treatment groups was: adjusted HR=0.88 (95% CI: 0.68-1.13; p=0.32) (FIG. 8A). The corresponding figures for OS were: adjusted HR=0.83 (95% CI: 0.64-1.08; p=0.17) (FIG. 8B). In contrast, comparing IDFS between CEF and CMF treated patients with lack of TIMP-1 cancer cell immunoreactivity showed an adjusted HR=0.51 (95% CI: 0.31-0.84; p=0.0085) (FIG. 8A) and OS adjusted HR=0.58 (95% CI: 0.35-0.96; p=0.03) (FIG. 8B) in favour of patients treated with CEF. A non-reduced Cox proportional hazards model was used to test for interactions between treatment effect and TIMP-1 with respect to IDFS and OS. A non-significant TIMP-1 profile (positive or negative immunoreactivity) versus treatment (CEF or CMF) interaction was detected for IDFS (p=0.06) (FIG. 8A) and OS (p=0.21) (FIG. 8B).

### Discussion

This study shows for the first time that lack of TIMP-1 cancer cell immunoreactivity is associated with a favourable effect of adjuvant epirubicin containing adjuvant therapy in primary breast cancer as compared with CMF, suggesting a predictive value of TIMP-1 immunoreactivity for anthracyclines. Compared with CMF, anthracycline based adjuvant treatment of TIMP-1 negative patients significantly reduces the risk of recurrence with 49% and mortality with 42%.

The VT7 anti-TIMP-1 monoclonal antibody was previously selected among a panel of anti-TIMP-1 antibodies for its superiority regarding immunostaining. VT7 recognizes a linear TIMP-1 epitope located between amino acid 169-174. The VT7 immunostaining was thoroughly validated with regard to sensitivity and specificity (VT7 does not bind TIMP-2, 3 or 4) and the staining conditions were optimized regarding antigen retrieval protocol, antibody concentration and time of incubation etc. In addition, the potential influence of fixation time (24-72 hours) was tested. On each TMA, a negative control antibody of the same IgG1 subtype (anti-TNP) was used and a slide of a known TIMP-1 positive breast cancer was included in each assay run as a positive control.

Only minor differences were observed in the characteristics of the 647 patients included in present analyses compared to the 980 Danish patients included in the original 89D trial, which indicates that the present 647 patients are representative for the whole DBCG 89D Danish study cohort. The overall benefits reported in the original 89D trial was reproduced in the present subset, which further support that the 647 patients are representative for the entire cohort of Danish patients in the DBCG trial 89D.

The present inventors have previously published that murine fibro sarcoma cells derived from TIMP-1 gene-deficient mice are significantly more sensitive to etoposide (a topoisomerase II inhibitor) in vitro than wild-type murine fibro sarcoma cells expressing TIMP-1. By applying an apoptosis assay, it was demonstrated that TIMP-1 protected the fibro sarcoma cells against apoptosis. That TIMP-1 can protect against chemotherapy-induced apoptosis has also been demonstrated by others. It is at present not clear why TIMP-1 in the present study predicts sensitivity/resistance to CEF and not to CMF. Suggestions have been made regarding the signalling pathways possibly regulated by TIMP-1. In the MCF10A breast epithelial cell line over-expression of TIMP-1 was shown to induce constitutive activation of focal adhesion kinase (FAK) through tyrosine phosphorylation. FAK has previously been shown to be upstream regulator of the phosphatidylinositol-3 kinase (PI-3 kinase) leading to regulation of the bcl-2 family members, a well-characterised signalling pathway leading to cell survival. Phosphorylated FAK associates with and thereby activates the PI-3 kinase, which in turn activates the Akt-kinase. Akt phosphorylates the protein Bad, which as a result is sequestered in the cytoplasm by the capture protein 14-3-3 and can therefore no longer interact with and inhibit bcl-2 and bcl-X_{L}. Bcl-2 and bcl-X_{L} are proteins situated in the mitochondrial membrane and when activated these anti-apoptotic proteins inhibit Bax thereby preventing the release of cytochrome c from the mitochondria. This in turn prevents activation of the caspase cascade and accordingly prevents apoptosis. Thus, TIMP-1 may inhibit apoptosis by acting like a trophic factor initiating the survival pathway including FAK, PI-3 kinase, Akt and bcl-2 family members resulting in inhibition of caspase activation and thereby inhibition of apoptosis.

By testing for TIMP-1 immunoreactivity in tumour tissue obtained from patients who were enrolled in the DBCG 89D trial, the present inventors have now shown that patients who lack TIMP-1 immunoreactivity in their breast cancer cells and who are treated with anthracycline containing combination chemotherapy have a significantly better outcome than patients treated with CMF. In the multivariate analyses, patients with TIMP-1 negative tumours had a 49% reduced risk of recurrence and 42% reduced risk of death when treated with CEF rather than with CMF. These clinical results are thus yet another support for our hypothesis that the TIMP-1 protein is associated with sensitivity/resistance to anthracycline treatment. However, an independent study is awaited to confirm the significant association between TIMP-1 immunoreactivity and anthracycline sensitivity/resistance in the adjuvant setting. Moreover, we are currently comparing the TIMP-1 results with those of HER2 and TOP2A gene aberration assays, both of which have been associated with sensitivity to anthracyclines.

The present inventors have previously published that the level of TIMP-1 protein in primary breast cancers carries prognostic information. It can thus be speculated whether the observed effect of TIMP-1 immunoreactivity on IDFS is prognostic or predictive. As no effect of TIMP-1 immunoreactivity was observed among CMF patients but only among CEF treated patients, the present results suggest that TIMP-1 immunoreactivity carries some predictive value and the present study is thus in line with our preclinical observations. In the prior prognostic studies, TIMP-1 protein was extracted from the whole tumour and the measured TIMP-1 protein could thus be derived from contaminating blood, from tumor tissue stromal cells, from extracellular matrix and from the cancer cells. In contrast, in the present study, only TIMP-1 protein localization in the epithelial cancer cells was included in the final analyses, which may be another reason for the differences between the present and the previous studies.

In conclusion, the present study, demonstrates for the first time that tumours being devoid of TIMP-1 protein immunoreactivity in the epithelial cancer cells are more sensitive to anthracycline treatment than to CMF treatment. Future studies will be aimed at establishing the relationship between TIMP-1 immunoreactivity, HER2, TOP2A and effect of anthracyclines. Moreover, the present results will be validated in an independent patient cohort.

### Example 2

### Clinical Study of the Combined Predictive Value of TOP2A and TIMP-1 Tumor Cell Gene Aberrations and TIMP-1 Tumor Cell Protein Immunoreactivity

### Methods

647 patient samples were obtained from a randomized study in which high risk breast cancer patients were randomized to adjuvant treatment with either CMF or CEF. End-point was invasive disease free survival (IDFS).

The patients samples consisted of tissue micro arrays made from the formalin fixed paraffin embedded tissue from the primary tumors of the patients. All samples had an identification number.

TOP2A gene aberrations were tested as previously described (Koop et al. 2005).

TIMP-1 gene aberrations were tested using standard FISH technology. BAC (Bacterial artificial chromosome) clone (RP11-466C12) was identified by analysis of a 400 kb area around the TIMP-1 gene using the UCSC genome browser (http://genome.ucsc.edu). The BAC clone is covering the previously identified genes; ARAF will-type allele (ARAF), human synapsin I (SYN1), tissue inhibitor of metalloproteinases-1 (TIMP-1), complement factor properdin (CFP), ELK1, ubiquitously expressed transcript (UXT), and AK094108. The clone was cultured in LB medium (Sigma Aldrich, Denmark) supplemented with 12.5 μg/mL chloramphenicol (Sigma Aldrich, Denmark) and purified according to the alkaline purification of BAC DNA (Poulsen 2004)(Poulsen T S, 2004). The clone was verified using in silico BamHI digest of the DNA sequence from the UCSC and compared with a BamHI endonuclease digestion of the purified BAC clone as recommended by the enzyme manufacture (Invitrogen, Denmark).

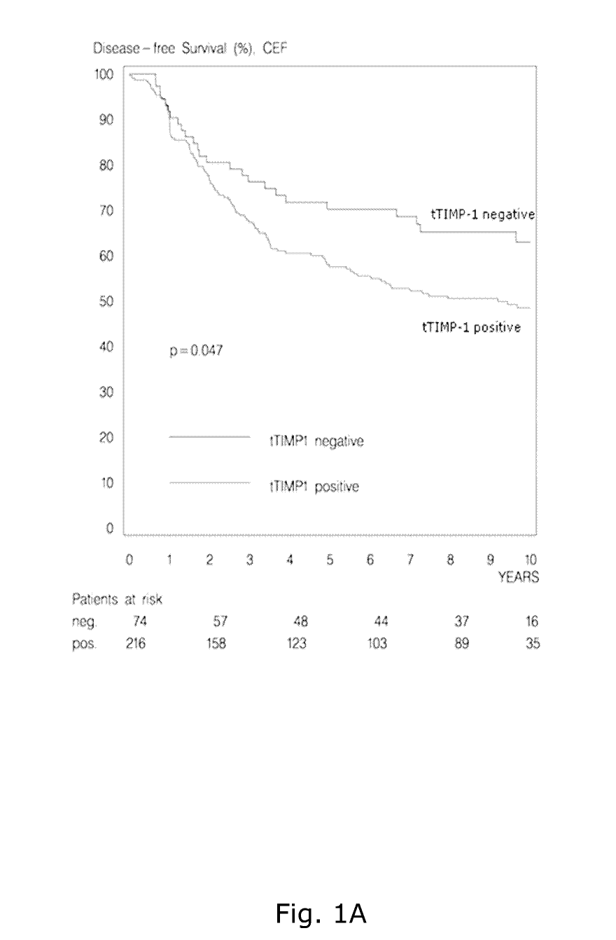

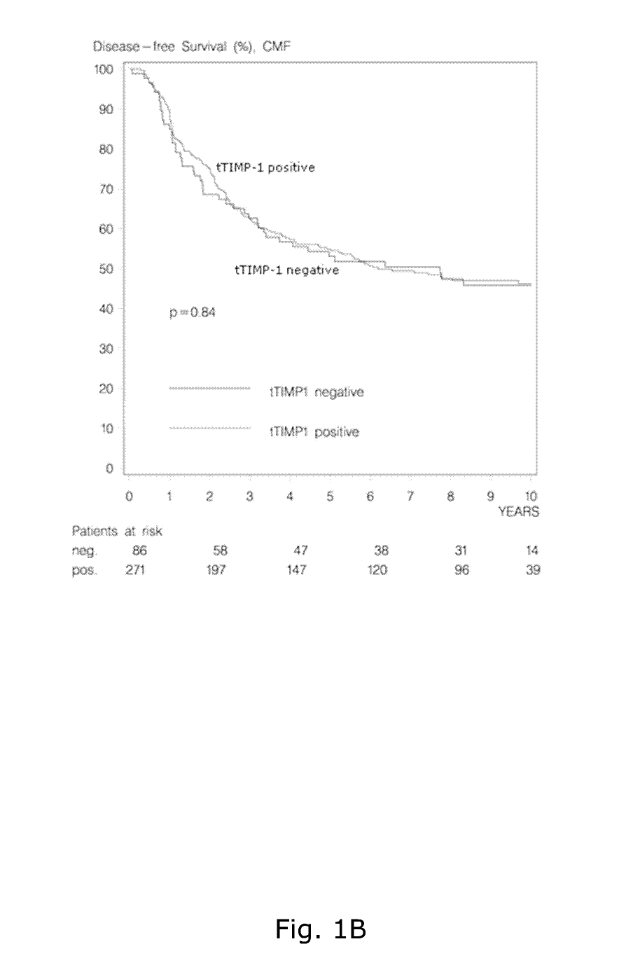

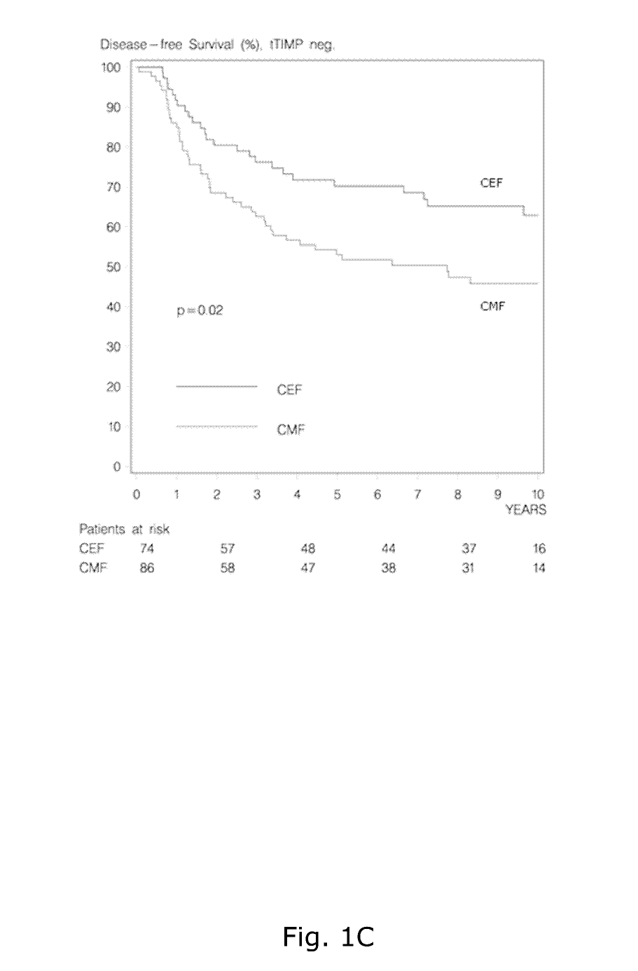

The probe BAC DNA was labeled by nick translation with Texas Red-5-dCTP (Millipore Corporation, Temecula, Calif., USA) as described by the manufacturer (Roche Diagnostics GmBH, Mannheim, Germany). A total of 10 ng/μL labeled DNA were used for FISH and suppression of undesired background staining derived from repetitive sequences was achieved using specific PNA oligos (Nielsen, K V et al., 2004). A fluoroscein labeled mixture of PNAs specific for the chromosome X α-satellite sequences (CenX PNA probe) was used as a reference for the copy number of chromosome X. The PNAs was supplied by Dako A/S. FIG. 1 shows a schematic representation of chromosome X and the localization of the part of region Xp11 covered by the BAC DNA as well as the area of centromere X covered by the CenX PNA probe. FISH was carried out using the Histology FISH accessory kit as described by the manufacturer (K5599, Dako A/S, Denmark), with modification. The pre-treatment step was not done by use of a water-bath but performed using a microwave oven (Whirlpool, Denmark, model JT356 with 6^{th} sense). Slides were submerged in enough 1× pre-treatment buffer to completely cover the slides, treated for 10 minutes using the steam function (6^{th} sense) followed by 15 minutes at room temperature (RT), before continuing according to the protocol supplied with the Histology FISH accessory kit.

### Evaluation of FISH

Hybridization signals were scored using a Leica microscope (Leica, Denmark) equipped with a 100×oil-immersion objective (numeric aperture). A dual-bandpass fluorescence filter (Chromotechnology, Brattleboro, Vt.) was used to visualize the FITC and Texas Red signals simultaneously. Sixty nonoverlapping interphase nuclei with intact morphology based on DAPI counterstaining were scored to determine the number of hybridization signals for each TIMP-1 and CenX probes. Amplification of TIMP-1 was defined as an average ratio of TIMP-1 signals relative to CenX signals (=level of amplification) of 2 or more (Ratio≧2). TIMP-1 was defined as deleted if the ratio was less than 0.8 (Ratio<0.8). Normal TIMP-1 gene/CenX ratio was therefore defined in between (0.8≦Ratio<2).

### Evaluation of TIMP-1 Immunoreactivity

Immunohistochemistry for the TIMP-1 protein was performed using the VT7 anti TIMP-1 monoclonal antibody (Sørensen et al. 2005) according to a previously published procedure (Sørensen et al 2005). The mouse monoclonal antibody (clone VT7, IgG₁) raised against recombinant human TIMP-1 (Moller Sorensen, et al. 2005; Sorensen, et al. 2006) was used at a concentration of 0.4 μg/ml.

All sections were evaluated by two independent pathologists who were unaware of the clinical history of the patients. Each sample was evaluated for presence or absence of tumor cell immunoreactivity and thus scores as either + or −.

All data were then transferred to the Danish Breast Cancer Cooperative Group Secretariat for statistical analyses.

### Results

290 patients had received CEF and 357 had received CMF. Of these, 216/290 and 271/357 were found positive for TIMP-1 immunoreactivity and 61/290 and 78/357 had TOP2 gene aberrations (amplifications or deletions). 24 patients had unknown TOP2A DNA status.

Kaplan Meier plots for disease free survival for patients stratified according to TIMP-1 tumor cell immunoreactivy is shown in FIGS. 1A and B. FIG. 1B shows that in the patients receiving CMF, TIMP-1 tumor cell reactivity had no impact on DFS (p=0.84). In contrast, in patients receiving CEF, lack of tumor cell TIMP-1 immunoreactivity was associated with a significant increased DFS (p=0.047) (FIG. 1A). In contrast, patients with TIMP-1 immunoreactivity in their tumor cells had a DFS comparable to patients treated with CMF (p=0.46).

As can bee seen from FIG. 1A, which shows the disease free survival of patients treated with CEF, patients absent of TIMP-1 immunoreactivity in the tumor cells do significantly better with regard to disease free survival. For example, at 5 years follow up, approximately 72% of the TIMP-1 negative patients have not experienced disease recurrence while only 60% of the TIMP-1 positive patients are free of disease.

FIG. 1B shows the disease free survival of patients receiving CMF and stratified according to whether the tumor cells display TIMP-1 immunoreactivity or not. There is no difference in disease free survival between the two groups.

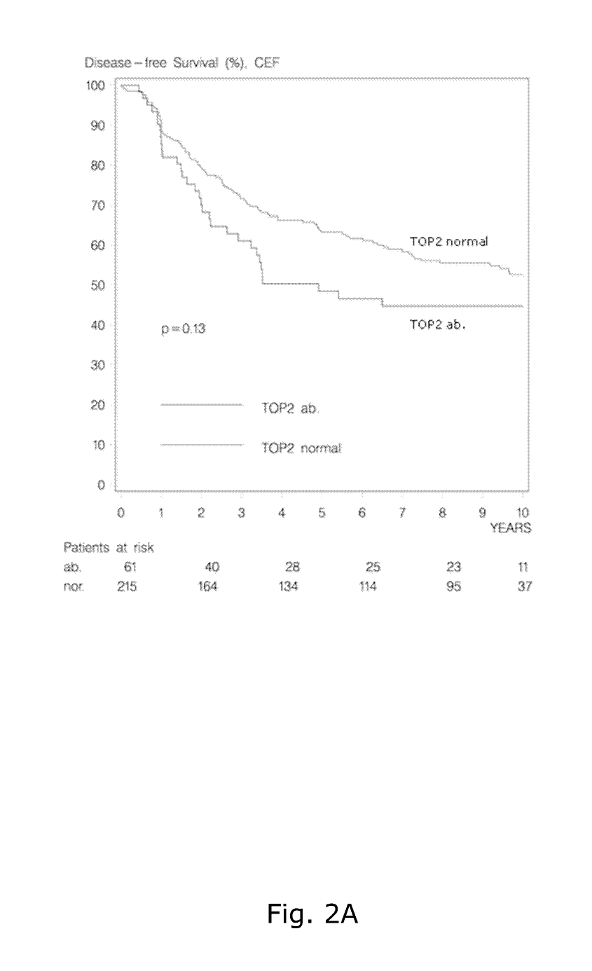

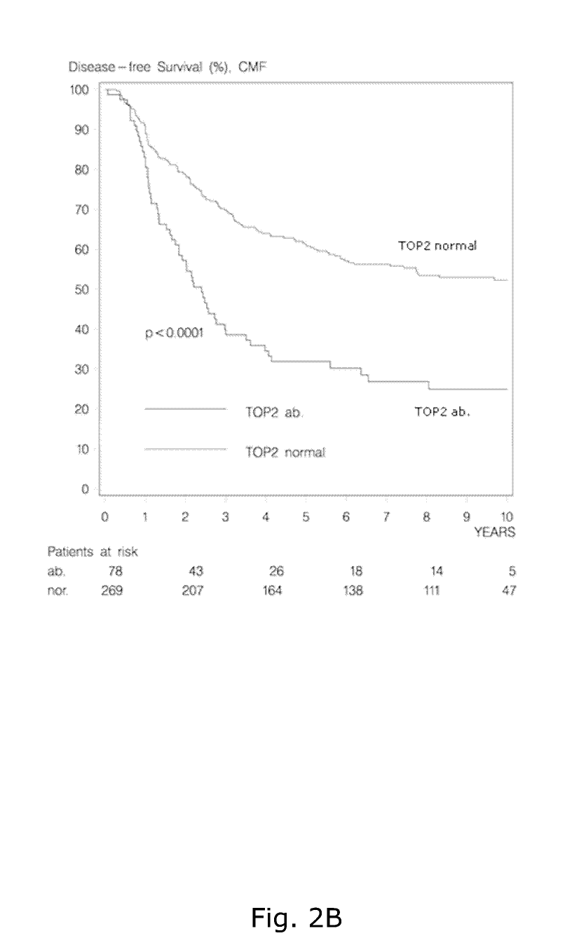

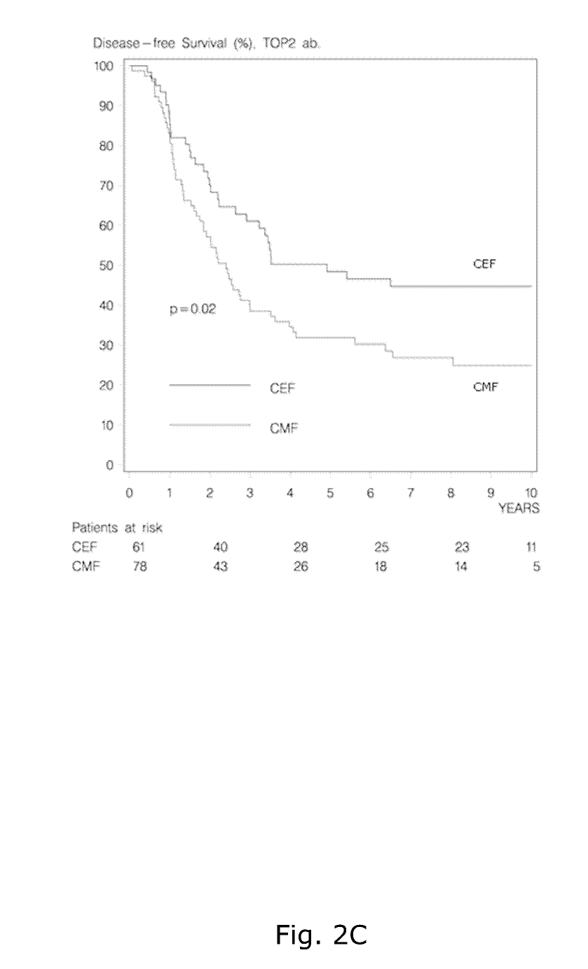

When analysing for TOP2A gene aberrations, it was found (FIGS. 2A and B) that in patients receiving CMF the TOP2A gene aberration status had no influence on DFS (FIG. 2B). In contrast, in patients receiving CEF, those patients with TOP2A gene aberrations (amplifications or deletions) had a significant improved DFS as compared to those patients with TOP2A DNA aberration who received CMF (FIG. 2A).

As can bee seen from FIG. 2B, which shows disease free survival of patients treated with CMF, patients with TOP2A DNA aberrations do much worse than patients without TOP2A DNA aberrations. However, when looking at FIG. 2A, which shows the disease free survival of patients receiving CEF and stratified for TOP2A DNA aberrations, it is seen that the curve (patient with TOP2A DNA aberrations) do better than those who received CMF (FIG. 2B)

It appeared that among the patients with negative TIMP-1 immunoreactivity in their cancer cells, only 24/160 (15%) had TOP2A gene aberrations. We therefore analysed the combined effect of having either TOP2A gene aberration or lack of TIMP-1 immunoreactivity on DFS. The results showed that it was now possible to identify almost the double number of patients with a high likelihood of obtaining benefit from CEF treatment (as compared with CMF treatment) as could be identified by TOP2A analyses alone and without reducing the hazard ratio. Table 1 shows the individual adjusted hazard ratios including 95% confidence intervals. All values are based on the CMF group being set to a hazard ratio of 1.

A HR of 1 means no difference between the groups. We have used the combined CMF groups as reference. Thus, the Table shows the benefit from CEF treatment compared to treatment with CMF in the subgroups.

It is seen from the Table 1 that patients with TOP2A DNA aberrations or TIMP-1 negativity treated with CEF have HR below 1 and that the 95% confidence intervals do not exceed 1. This means that these patients (TOP2A DNA aberrations and/or TIMP-1 negativity) benefit significantly more from the CEF treatment as compared with the treatment with CMF. A HR of 0.54 means that chance of benefit for the patients (TOP2A DNA aberrations and/or TIMP-1 negativity) is 46%. It is also seen from the Table, that the HR for TOP2A DNA aberrations (amplifications or deletions) and for patients who's tumor cells are absent of TIMP-1 immunoreactivy have almost similar HR. The invention is that it is not always the same patients having TOP2A DNA aberrations or being absent of TIMP-1 protein immunoreactivy. Then when looking at the HR for the group of patients with TOP2A DNA aberrations and/or absent of TIMP-1 immunoreactivity, the HR stays almost the same (0.48 (95% confidence interval: 034-069) despite the number of patients in this subgroup is almost double up of the number of patients that could be identified by TOP2A DNA aberrations alone. In other words, by combining TOP2A DNA aberration measurements with TIMP-1 protein immunoreactivy measurements, almost double as many patients that have a high likelihood of benefit from CEF is identified as compared to TOP2A DNA aberration measurements alone.

By the combined method it is possible to identify 43% of the patients who had more than 50% increased likelihood of obtaining benefit from CEF treatment as compared with the benefit from CMF treatment (Hazard ratio 0.48) which is approximately the double number of what can be accomplished by analysing only for TOP2A DNA aberrations alone.

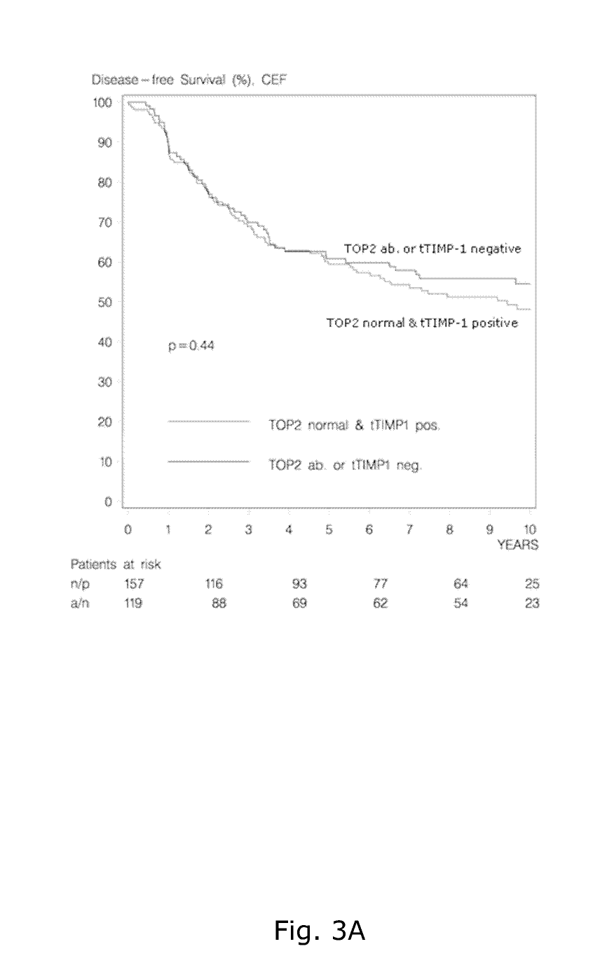

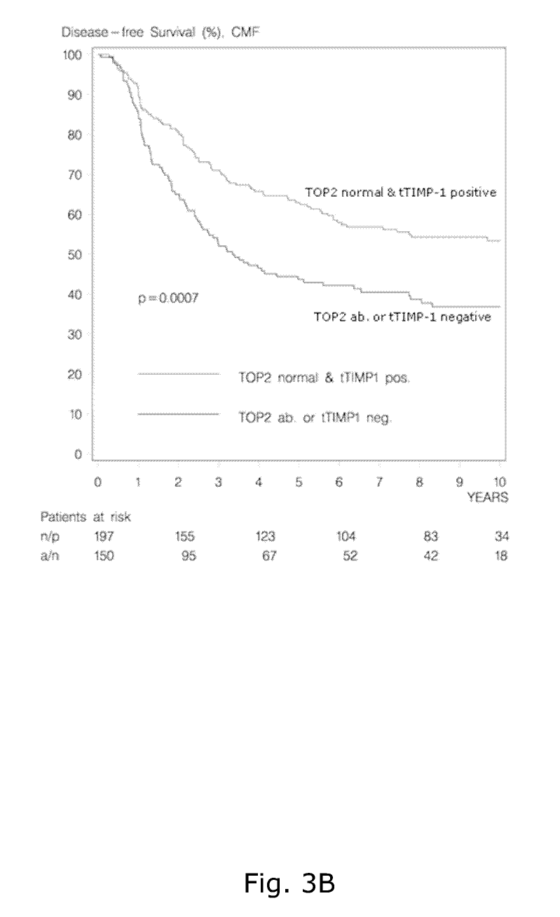

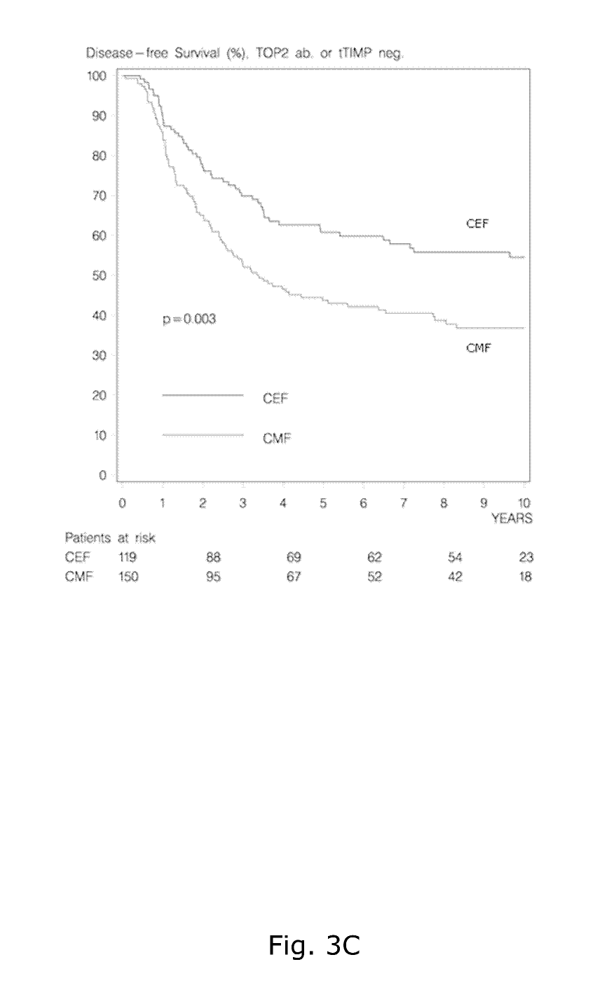

FIGS. 3A and B show the Kaplan Meir curves for DFS when TOP-2A DNA aberrations and TIMP-1 immunoreactivity is combined.

When looking at FIG. 3B, it is seen that patients with TOP2A DNA aberrations and/or absence of tumor cell TIMP-1 protein immunoreactivity do worse than patients without TOP2A DNA aberrations and with TIMP-1 protein immunoreactivity in their tumor cells when treated with CMF. However, if the patients are treated with CEF (FIG. 3A), the patients with TOP2A DNA Aberrations and/or lack of TIMP-1 protein immunoreactivity do much better than those treated with CMF. Thus, patients with TOP2A DNA aberrations and/or lack of TIMP-1 protein immunoreactivity and treated with CEF do better than patients with TOP2A DNA aberrations and/or lack of TIMP-1 protein immunoreactivity treated with CMF.

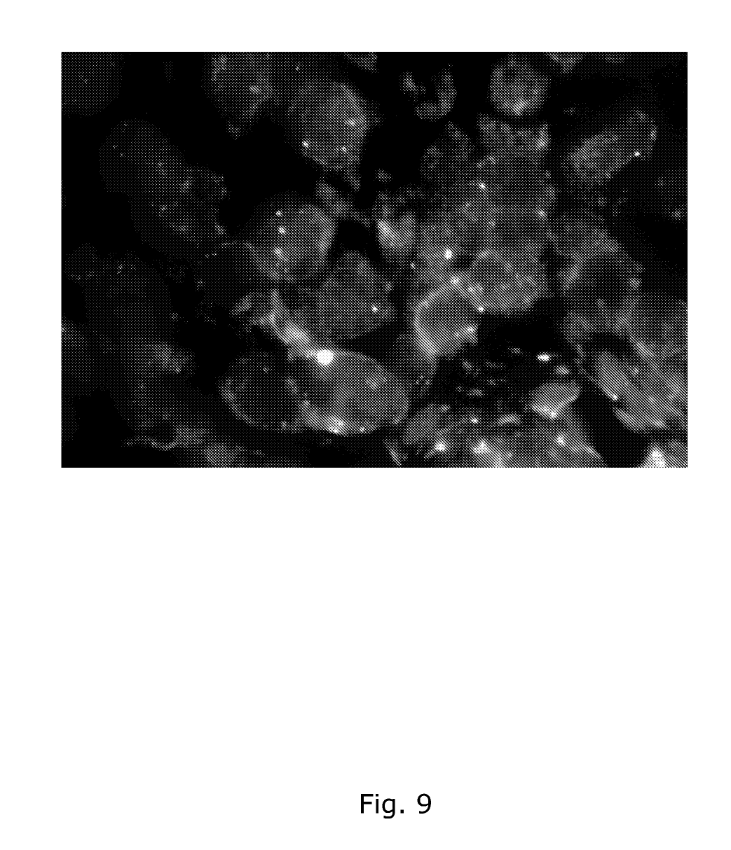

FIG. 9 shows TIMP-1 FISH analysis showing TIMP-1 DNA amplifications in epithelial breast cancer cells

### Discussion

This study demonstrates that lack or reduced concentration of TIMP-1 protein and/or TOP2A gene aberrations confers sensitivity to certain types of chemotherapy.

The present study was performed on samples obtained from a large prospective study with full clinical follow up (Ejlertsen et al., Eur J Cancer 2005). Both the TOP2A FISH analyses and the TIMP-1 immunohistochemistry technologies used have previously been described.

The results of the present study clearly demonstrate the additive effect of combining TOP2A gene aberration measurements with TIMP-1 immunohistochemistry in predicting benefit (prolonged IDFS) from adjuvant treatment with CEF in primary high risk breast cancer patients while no benefit is observed in patients treated with CMF, suggesting the value of the combined test in predicting benefit from anthracycline containing chemotherapy.

### Example 3

### HER2, TOP2A and TIMP-1 and Responsiveness to Adjuvant Anthracycline Containing Chemotherapy in High Risk Breast Cancer Patients

### Methods

The DBCG 89D trial and its biological sub-study has previously been described in detail (Ejlertsen at al. 2007 and Knoop et al. 2005). Briefly, DBCG trial 89D is an open-labeled randomized, phase III trial comparing CEF (cyclophosphamide 600 mg/m², epirubicin 60 mg/m², and fluorouracil 600 mg/m²) against CMF (cyclophosphamide 600 mg/m², methotrexate 40 mg/m², and fluorouracil 600 mg/m²) both intravenously for nine cycles with 3 week intervals. Eligible for the 89D trial were patients' with hormone receptor negative and node positive (or tumor size>5 cm) breast cancer, and premenopausal patients with node negative tumors provided they had malignancy grade II or III. Patients with highly hormone responsive tumors were included in DBCG trials, 89B and 89C, with synchronized eligibility criteria. The DBCG prepared the original protocol as well as the biomarker supplements and The Danish National Committee on Biomedical Research Ethics approved the original protocol as well as the supplements before their activation (V.200.1616/89, KF 12 295 003).

Central assessment of HER2, ER AND TIMP-1 immunoreactivity Tissue microarrays (TMA) were constructed from formalin-fixed and paraffin-embedded tumor blocks by means of a TMA-builder (Histopathology Ltd, AH-diagnostics). A target area was identified in the donor block on haematoxylin stained sections and two 2 mm tissue cores were transferred to the recipient TMA block. ER immunostaining was performed at room temperature on 3μ TMA sections with the ER1D5 (Dako) antibody and a Tech-mate 500 (Dako). ER expression was recorded as the percentage of staining tumor cells, ignoring intensity, and the results were dichotomized as positive (≧10% staining cells) or negative (<10%). Expression of HER2 was measured on whole sections using the HercepTest (Dako) and scored accordingly as 0, 1+, 2+, or 3+. TIMP-1 immunostaining was performed as previously described (Sorensen et al. 2006). In brief, sections were incubated with the anti TIMP-1 mouse monoclonal antibody VT7. VT7 was detected with mouse/rabbit Envision+ (Code No K5007, DAKO A/S), and the reaction was visualized by incubating the sections with DAB+ (Code No K5007, DAKO A/S) for 2 periods of 3 minutes. Immunostaining of tissue sections was assessed semi-quantitatively using + and − symbols as a measure of TIMP-1 immunoreactivity in the epithelial breast cancer cells. Scoring of the intensity of the signal was not included. The scoring of the tissue sections was performed blinded by two independent pathologists (GW and EB). In case of discrepancies, agreement was reached by looking at the slides together.

### TOP2A and HER2 FISH

TOP2A and HER2 copy number was visualized by FISH (TOP2A pharmDX and HER2 pharmDX, DAKO A/S). At least 60 gene signals were scored and all signals were scored if a nucleus was included. The centromere 17 signals were in addition scored in the same nuclei's, and the ratio of gene to centromere 17 was calculated. Tumors were scored as TOP2A/HER2 deleted, normal or amplified according to a ratio of <0.8, 0.8-1.9 and >2.0.

### Statistical Methods

Follow-up time was quantified in terms of a Kaplan-Meier estimate of potential follow-up. Invasive Disease-Free Survival (IDFS) was the primary end-point and was defined as the time elapsed from randomization until invasive breast cancer recurrence irrespective of localization, invasive breast cancer involving the same or the contralateral breast, second primary non-breast invasive cancer or death attributable to any cause. Overall survival (OS), the secondary end-point, was defined as the elapsed time from randomization until death attributable to any cause. IDFS and OS were analyzed using Kaplan-Meier estimates and the logrank test. The effect of TIMP-1 in combination with HER2 or TOP2A biomarker status on IDFS and OS was quantified in terms of the hazard ratio, estimated unadjusted using the Cox proportional hazards model. The Cox proportional hazards model was also applied for multivariate analysis, based on the model developed previously for the same patient material. The multivariate model included TIMP-1, TOP2A, HER2, ER, tumor size, positive lymph nodes, histologic type and grade, menopausal status, and treatment with CMF or CEF. The Cox proportional hazards model on IDFS and OS was adjusted according to the results of the goodness-of-fit procedures, and ER hormone receptor status as well as histological type and grade were included as stratification variables. Interaction between biomarkers (HT, 2T, TIMP-1, TOP2A, and HER2) and treatment regimens (CMF or CEF) were investigated in separate models, and the Wald Test was applied.

Differences between patients with and without information about biomarkers, between treatment regimens, and correlations between HT (HER2 positive and/or lack of TIMP-1 immunoreactivity) or 2T biomarker status and clinical and pathological variables including HER2-status were tested (excluded unknowns) by χ2-test. P-values are two-tailed. Tumors were classified as HT responsive if HER2 positive and/or lack of TIMP-1 immunoreactivity and otherwise HT non-responsive. Tumors were classified as 2T responsive if they had TOP2A aberrations and/or lack of TIMP-1 immunoreactivity and otherwise 2T non-responsive. Statistical analyses were done with the SAS 9·1 program package.

The DBCG was responsible for study design and coordination, tissue collection, biomarker analysis, data collection, analysis, and reporting. The ER1D5 antibody, HercepTest, HER2 phamDX and TOP2A phamDX kits and technical assistance were provided free of charge by DAKO A/S (Glostrup, Denmark).

### Results

The DBCG 89D trial recruited 1224 patients between June 1990 and January 1998. Median estimated potential follow-up was 9.8 years for IDFS and 13.8 years for OS. In 2001, the DBCG completed the retrospective collection of formalin-fixed, paraffin-embedded primary breast tumor tissue blocks that were available from 821 (84%) of the 980 participants enrolled in Denmark and the construction of TMA was successful in 708 patients (72%). A total of 623 patients were accessible for HER2, TOP2A and TIMP-1 analyses. The assessable 623 patients differed significantly from the 357 non-assessable (p<0.05) with regard to menopausal status, tumor size, malignancy grade, and ER status. Number of positive lymph nodes and histological type showed no significant differences between assessable and non-assessable patients. The treatment effect was similar, with a hazard ratio favoring CEF for IDFS (adjusted hazard ratio, 0.80 (95% confidence interval (CI), 0.63 to 1.01; P=0.06) and OS (adjusted hazard ratio, 0.79; 95% CI, 0.62 to 1.00; P=0.05) to the effect observed in the original study (IDFS: hazard ratio 0.76 and OS: hazard ratio 0.73)(Ejlertsen et al. 2007).

Among the accessible 623 patients 188 (30%) had a HER2 positive, 139 (22%) a TOP2A abnormal and 154 (25%) a TIMP-1 negative tumor. A TOP2A aberration was only detected in 33 (8%) of the 435 HER2 negative patients (Table 2). In contrast, TIMP-1 immunoreactivity was detected in 123 (28%) of the HER negative and in 130 (27%) of the 484 TOP2A normal patients. Table 2 shows the baseline characteristics according to 2T status for the 623 patients for whom HER2, TOP2A and TIMP-1 was successful performed.

Integrating TIMP-1 with TOP2A or HER2

By means of HER2 and TIMP-1 311 (50%) patients were classified as HT anthracycline responsive, e.g. had a HER2 positive, a TIMP-1 negative or a HER2 positive and TIMP-1 negative tumour profile. Patients with a HT responsive profile significantly more often (P<0.05) were postmenopausal, and had positive lymph nodes, tumors larger than 2 cm and ER negative tumours. Patients who had a HT responsive profile had a similar IDFS (hazard ratio, 1.22; 95% CI, 0.97 to 1.52; P=0.09) and inferior OS (hazard ratio, 1.33; 95% CI, 1.06 to 1.67; P=0.01) compared to those whose tumors were HT non-responsive. Adjustment for menopausal status, tumor size, number of positive lymph nodes, histologic type and grade, ER and TOP2A status, and treatment in a multivariate analysis changed the hazard ratio for IDFS (hazard ratio, 1.03; 95% CI, 0.80 to 1.33; P=0.81) and OS (hazard ratio, 1.05; 95% CI, 0.81 to 1.36; P=0.73).

With the integrated use of TOP2A and TIMP-1 269 (43%) patients were classified as 2T anthracycline responsive, e.g. had a TOP2A aberration and/or lacked TIMP-1 immunoreactivity (Table 2). A 2T responsive profile was associated with ER negativity, HER2 positivity and larger tumor size (all P<0.01). Patients with a 2T responsive profile had a decreased IDFS (hazard ratio, 1.26; 95% CI, 1.01 to 1.58; P=0.04) and OS (hazard ratio, 1.34; 95% CI, 1.07 to 1.69; P=0.01) as compared to those with a 2T non-responsive profile. Adjustment in a multivariate analysis for menopausal status, tumor size, number of positive lymph nodes, histologic type and grade, ER expression and HER2 status, and treatment changed the hazard ratio for IDFS (1.19; 95% CI, 0.93 to 1.51; P=0.71) and OS (1.18; 95% CI, 0.927 to 1.51; P=0.18).

### Heterogeneity of Treatment According to Single Biomarkers and Profiles

In the multivariate Cox regression analysis we further examined heterogeneity of treatment effect according to HER2 status, TOP2A status, TIMP-1 immunoreactivity, HT profile or 2T profile. There was no statistically significant interaction showing improved IDFS and OS with CEF compared with CMF for HER2 and TIMP-1. As was previously reported a significant interaction between TOP2A status and treatment effect was observed for IDFS (P=0.004) and OS (P=0.03).

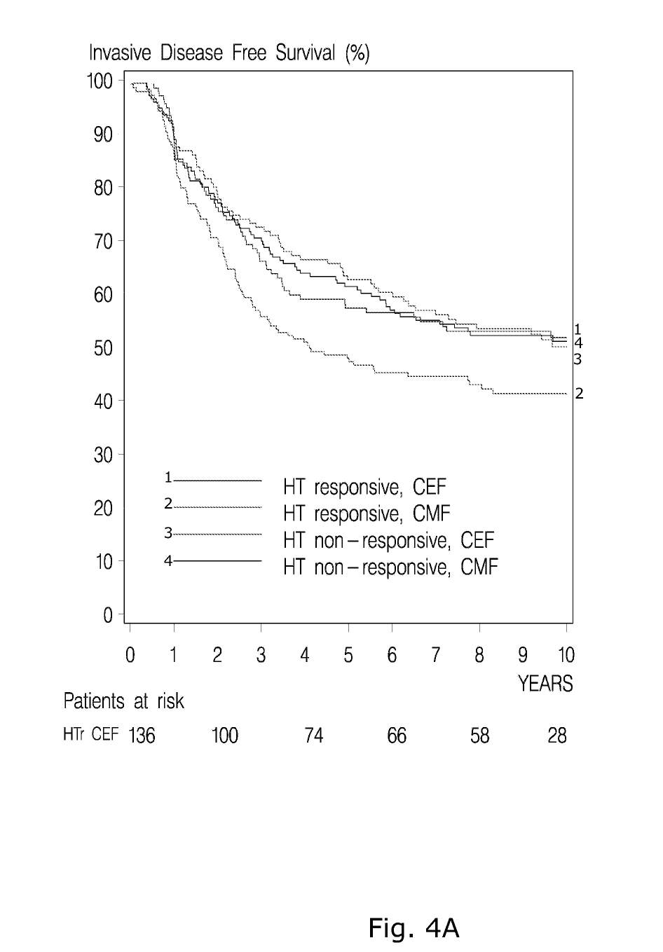

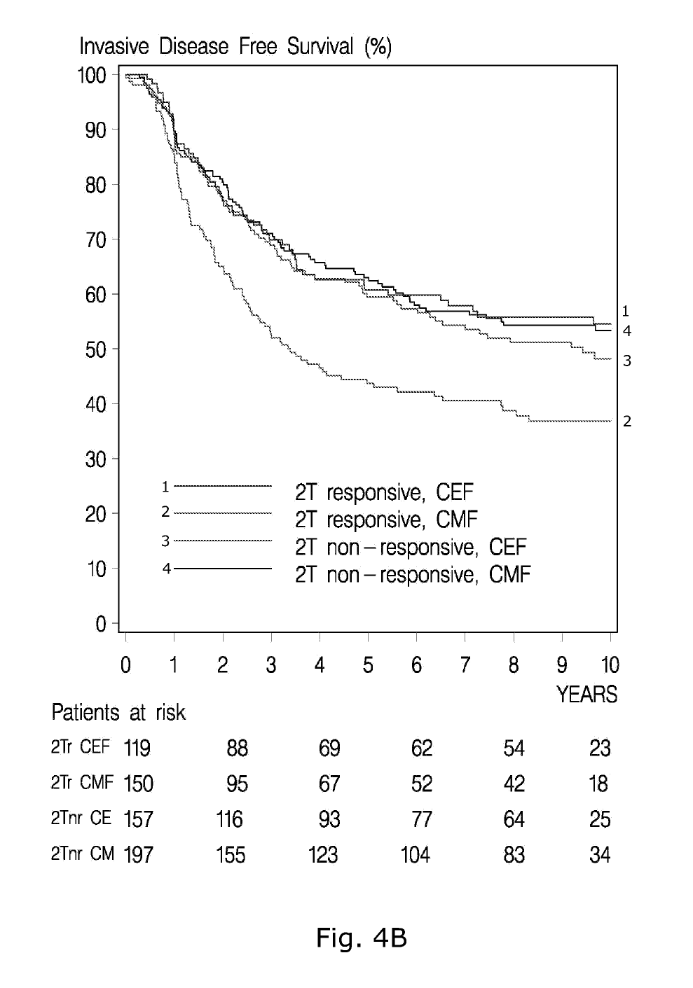

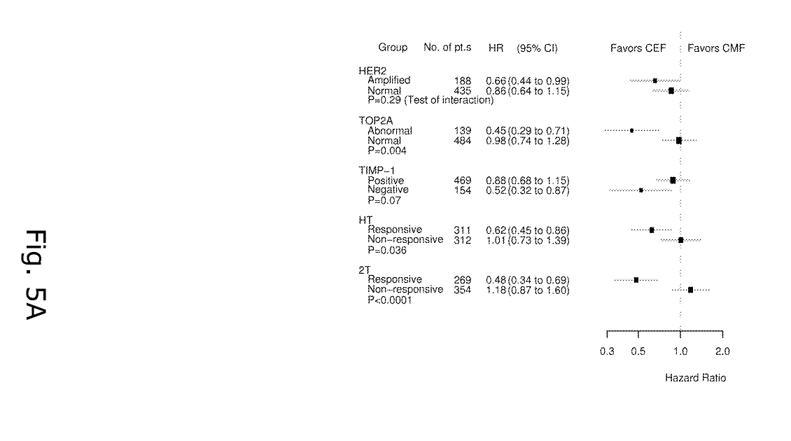

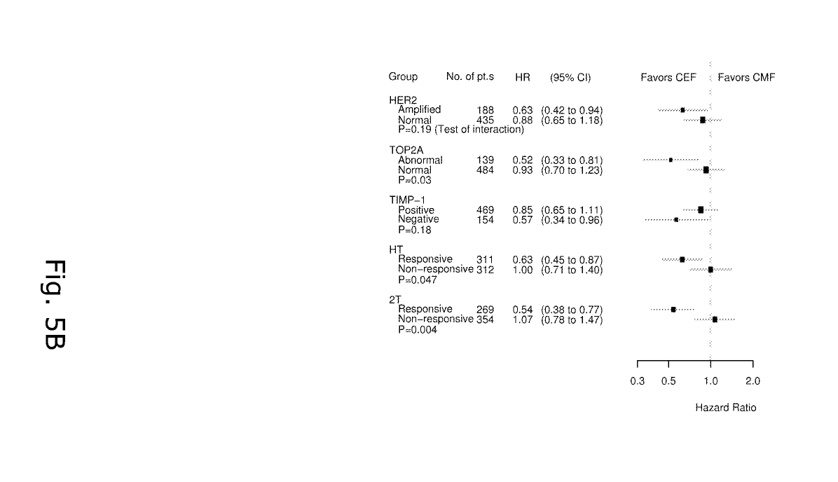

If treated with CEF, patients with tumors classified as HT responsive (HER2 positive or TIMP-1 negative) had a borderline significant improvement in IDFS (FIG. 4A, Table 4) and a statistically significant improvement in OS. By contrast, no significant benefit from CEF as compared to CMF was observed among patients with a HT non-responsive profile. A more favorable IDFS and OS with the use of CEF in patients with a HT responsive profile was sustained after adjustment for nodal status, tumor size, histology, grade, ER status, TOP2A status, HER2 status, TIMP-1 expression and menopausal status (P values=0.036 and 0.047, respectively; FIG. 5).

Among patients with a 2T responsive profile CEF significantly improved IDFS and OS compared with CMF (FIG. 4B, Table 4), as opposed to 2T non-responsive patients. A multivariate analysis adjusting for patient and tumor characteristics confirmed that patients with a 2T responsive profile benefited from CEF compared to CMF regarding both IDFS (FIG. 5A) and OS (FIG. 5B). A non-significant trend for a more favorable outcome with the use of CMF existed by contrast, in patients with a 2T non-responsive profile (FIG. 5). There was a highly statistically significant interaction between the 2T profile and treatment effect were the 269 (43%) patients with a 2T responsive (TOP2A aberration or TIMP-1 negative) profile experienced a more favorable outcome with the use of CEF compared to CMF regarding IDFS (Wald test, P<0.0001) and OS (Wald test, P=0.004) (FIG. 5).

### Discussion

In general it has been acknowledged, that the selection of therapies should whenever possible be directed against specific targets within the tumor of each individual breast cancer patient. The addition of chemotherapy is however often required and chemotherapy has been considered less target specific. Despite the demonstration of their superiority in the adjuvant setting the mechanism of action of anthracyclines is still not fully elucidated. Among the proposed mechanisms, interaction with topoisomerase II-a and induction of apoptosis however seems to occur at clinically relevant anthracycline concentrations.

The present inventors engaged in the development of a combined TOP2A and TIMP-1 profile and have previously examined their predictive properties individually within the DBCG 89D trial.

In the present study, among 188 patients with HER2 positive tumors 106 (56%) had abnormal TOP2A status, compared to 8% (33 of 435) with HER2 negative tumors. As a large number of patients with TOP2A abnormal tumors are contained within the HER2 positive population it was not feasible to combine these two markers. By integration of TOP2A and TIMP-1 in the 2T profile 43% of the patients were classified as anthracycline responsive compared to 22% using TOP2A and 25% using TIMP-1 alone. For the 43% of patients with a 2T responsive profile the use of CEF was associated with a relative reduction in IDFS events of 52% and a 46% relative reduction in mortality.

In contrast, a non-significant benefit from CMF was seen in the remaining 57% patients with a 2T non-responsive profile. The magnitude of difference among patients with a 2T responsive and non-responsive profile and the accuracy of these estimates are high enough to emphasize a clinical important difference. The finding of a highly statistically significant interaction between treatment and the 2T profile supports this statement. The 4% who had a TOP2A and a TIMP-1 responsive profile did not seem to have a different outcome.

HER2 is the most frequent used biomarker regarding sensitivity to anthracyclins, and the majority of TOP2A aberrations are observed among HER2 positive tumors. For comparison the present inventors combined HER2 and TIMP-1, and classified patients as HT anthracyclin responsive if the tumor lacked TIMP-1 immunoreactivity and/or were HER2 positive.

The benefit from CEF as compared to CMF was substantially larger in the 50% of patients with a HT responsive profile, and this heterogeneity was confirmed by a statistically significant interaction between the HT profile and treatment. The present inventors did not find evidence for a differential treatment effect according to TIMP-1 or HER2 as single markers, which emphasis the power of integrating biomarkers.

In conclusion, the combined analysis of the 2T profile based on both TOP2A and TIMP-1 show that in combination these two biomarkers identify the greater part, if not nearly all patients who benefits significantly from substituting methotrexate in CMF with epirubicin. The 2T profile separates out a larger anthracycline responsive subgroup than HER2, TOP2A and TIMP-1 do individually.

### Tables

TABLE 1

| | Hazard ratio | 95% confidence intervals |
|---|---|---|
| TOP2A DNA deletion | 0.53 | 0.28-1.0 |
| TOP2A DNA amplification | 0.38 | 0.2-0.72 |
| TIMP-1 lack of | 0.54 | 0.31-0.93 |
| immunoreactivity in tumor | | |
| cells in patients without | | |
| TOP2A gene aberrations | | |
| TOP2A DNA aberrations or | 0.48 | 0.34-0.69 |
| lack of TIMP-1 | | |
| immunoreactivity in the | | |
| cancer cells | | |
| Lack of TOP2A DNA | 1.19 | 0.87-1.61 |
| aberrations or positive | | |
| TIMP-1 immunoreactivity | | |
| in the cancer cells | | |

TABLE 2
Distribution of TIMP-1 Immunoreactivity According
to HER2 and TOP2A Status.

| | TOP2A abnormal | | | | TOP2A normal | | | | |
|---|---|---|---|---|---|---|---|---|---|
| | HER2 | | HER2 | | HER2 | | HER2 | | |
| | positive | | negative | | positive | | negative | | |
| TIMP-1 | N | % | N | % | N | % | N | % | Total |
| Positive | 89 | 14 | 26 | 4 | 68 | 11 | 286 | 46 | 469 |
| Negative | 17 | 3 | 7 | 1 | 14 | 2 | 116 | 19 | 154 |
| Total | 106 | 17 | 33 | 5 | 82 | 13 | 402 | 65 | 623 |

TABLE 3
Baseline Characteristics According to 2T Profile

| | Responsive | | Non-responsive | | |
|---|---|---|---|---|---|
| | profile | | profile | | |
| | (N = 269) | | (N = 354) | | |
| Characteristic | No. | (%) | No. | (%) | P Value |
| Menopausal status | | | | | P = 0.0497 |
| Premenopausal | 174 | 65 | 255 | 72 | |
| Postmenopausal | 95 | 35 | 99 | 28 | |
| Local-regional therapy | | | | | P = 0.04 |
| Breast | 36 | 13 | 70 | 20 | |
| conserving | | | | | |
| Mastectomy | 233 | 87 | 284 | 80 | |
| Estrogen receptor status | | | | | P = 0.004 |
| Positive | 69 | 26 | 128 | 36 | |
| Negative | 184 | 68 | 203 | 57 | |
| Unknown | 16 | 6 | 23 | 7 | |
| HER2 status | | | | | P < 0.0001 |
| Positive | 120 | 45 | 68 | 19 | |
| Negative | 149 | 55 | 286 | 81 | |

| Positive nodes | | | Positive nodes | |
|---|---|---|---|---|
| None | 89 | 33 | None | 89 |
| 1-3 | 86 | 32 | 1-3 | 86 |
| >3 | 94 | 35 | >3 | 94 |
| Tumor size, millimeters | | | Tumor size, millimeters | |
| 0-20 | 88 | 33 | 0-20 | 88 |
| 21-50 | 152 | 57 | 21-50 | 152 |
| >50 | 28 | 10 | >50 | 28 |
| Unknown | 1 | 0 | Unknown | 1 |
| Malignancy grade | | | Malignancy grade | |
| Grade I | 14 | 5 | Grade I | 14 |
| Grade II | 124 | 46 | Grade II | 124 |
| Grade III | 113 | 42 | Grade III | 113 |
| Unknown | 1 | 0 | Unknown | 1 |
| Non-ductal | 17 | 6 | Non-ductal | 17 |
| Treatment | | | Treatment | |
| CMF | 150 | 56 | CMF | 150 |
| CEF | 119 | 44 | CEF | 119 |

TABLE 4
Unadjusted hazard ratio estimates of treatment effect for IDFS
and OS in HT and 2T Responsive and Non-responsive tumors.

| | IDFS | | | OS | | |
|---|---|---|---|---|---|---|
| | HR | (95% CI) | P | HR | (95% CI) | P |
| HT profile | | | | | | |
| Responsive | 0.73 | (0.53-1.00) | 0.05 | 0.69 | (0.50-0.95) | 0.02 |
| Non-responsive | 0.98 | (0.71-1.37) | 0.92 | 0.92 | (0.66-1.29) | 0.64 |
| 2T profile | | | | | | |
| Responsive | 0.59 | (0.42-0.83) | 0.003 | 0.63 | (0.45-0.88) | 0.007 |
| Non-responsive | 1.12 | (0.83-1.53) | 0.46 | 0.95 | (0.69-1.30) | 0.74 |

TABLE 5
Base-Line Characteristics of the Danish
Intention to Treat Population (n = 980)

| | |
|---|---|
| Excluded | Included |
| N = 333 | N = 647 |
| (34%) | (66%) |
| No. (%) | No. (%) |

| | | | | |
|---|---|---|---|---|
| Age at enrolment | | | | |
| ≦39 Years | 65 | 20 | 99 | 15 |
| 40-49 Years | 165 | 50 | 316 | 49 |
| 50-59 Years | 57 | 17 | 149 | 23 |
| 60-69 Years | 46 | 14 | 83 | 13 |
| Menopausal status | | | | |
| Premenopausal | 246 | 74 | 450 | 70 |
| Postmenopausal | 87 | 26 | 197 | 30 |
| Nodal status | | | | |
| Negative | 121 | 36 | 233 | 36 |
| 1-3 positive | 122 | 37 | 206 | 32 |
| ≧4 positive | 90 | 27 | 208 | 32 |
| Tumour size * | | | | |
| 0-20 mm | 179 | 55 | 253 | 39 |
| 21-50 mm | 130 | 40 | 336 | 52 |
| >50 mm | 19 | 6 | 56 | 9 |
| Unknown | 5 | 2 | 2 | 0 |
| Histologic type | | | | |
| Infiltrating ductal carcinoma | 313 | 94 | 602 | 93 |
| Other carcinomas | 17 | 5 | 44 | 7 |
| Unknown | 3 | 1 | 1 | 0 |
| Malignancy grade | | | | |
| (ductal carcinomas only) ** | | | | |
| Grade I | 27 | 9 | 43 | 7 |
| Grade II | 177 | 57 | 298 | 50 |
| Grade III | 104 | 33 | 259 | 43 |
| Unknown | 5 | 2 | 2 | 0 |
| Estrogen-receptor status | | | | |
| Positive | 7 | 2 | 199 | 31 |
| Negative | 26 | 8 | 401 | 62 |
| Unknown | 300 | 90 | 47 | 7 |
| Hormone-receptor status | | | | |
| ER or PgR positive | 88 | 26 | 167 | 26 |
| ER and PgR negative | 201 | 60 | 431 | 67 |
| Unknown | 44 | 13 | 49 | 8 |
| Chemotherapy | | | | |
| CMF | 158 | 47 | 357 | 55 |
| CEF | 157 | 47 | 290 | 45 |
| None | 18 | 5 | 0 | 0 |

p < 0.000.1;
** p = 0.02

TABLE 6
Base-Line Characteristics in relation to TIMP-1

| | |
|---|---|
| TIMP1 neg. | TIMP1 pos. |
| (N = 160) | (N = 487) |
| No. (%) | No. (%) |

| | | | | |
|---|---|---|---|---|
| Age at enrolment | | | | |
| ≦39 Years | 26 | (16) | 73 | (15) |
| 40-49 Years | 78 | (49) | 238 | (49) |
| 50-59 Years | 36 | (23) | 113 | (23) |
| 60-69 Years | 20 | (13) | 63 | (13) |
| Menopausal status | | | | |
| Premenopausal | 118 | (74) | 332 | (68) |
| Postmenopausal | 42 | (26) | 155 | (32) |
| Nodal status | | | | |
| Negative | 72 | (45) | 161 | (33) |
| 1-3 positive | 44 | (28) | 162 | (33) |
| ≧4 positive | 44 | (28) | 164 | (34) |
| Tumour size | | | | |
| 0-20 mm | 62 | (39) | 191 | (39) |
| 21-50 mm | 81 | (51) | 255 | (52) |
| >50 mm | 16 | (10) | 40 | (8) |
| Unknown | 1 | (1) | 1 | (0) |
| Histologic type | | | | |
| Infiltrating ductal carcinoma | 146 | (91) | 456 | (94) |
| Other carcinomas | 14 | (9) | 31 | (6) |
| Malignancy grade | | | | |
| (ductal carcinomas only) | | | | |
| Grade I | 9 | (6) | 34 | (7) |
| Grade II | 66 | (45) | 232 | (51) |
| Grade III | 70 | (48) | 189 | (41) |
| Unknown | 1 | (1) | 1 | (0) |
| Estrogen-receptor status | | | | |
| Positive | 38 | (24) | 161 | (33) |
| Negative | 107 | (67) | 294 | (60) |
| Unknown | 15 | (9) | 32 | (7) |
| Hormone-receptor status | | | | |
| ER or PgR positive | 36 | (23) | 131 | (27) |
| ER and PgR negative | 115 | (72) | 316 | (65) |
| Unknown | 9 | (6) | 40 | (8) |
| Chemotherapy | | | | |
| CMF | 86 | (54) | 271 | (56) |
| CEF | 74 | (46) | 216 | (44) |

TABLE 7
Diagram showing the patient flow

| | CMF | CEF |
|---|---|---|
| Cumulative allocation | 500 | 480 |
| Cross-over, self-selected CMF | +18 | −18 |
| Cross-over, self-selected CEF | −4 | +4 |
| Withdraw consent to chemotherapy | −5 | −13 |
| TIMP-1 unknown* | −152 | −163 |
| Included in the analyses | 357 | 290 |

*Archival tissue not available, tissue unsuited for TMA, tissue lost after TMA or TIMP-1 not assessable.

### REFERENCES

Louise Davidsen, Sidse Würtz, Maria Unni Rømer, Nanna M Sørensen, Simon Johansen, Ib Jarle Christensen, Jørgen K Larsen, Hanne Offenberg, Nils Brünner and Ulrik Lademann. TIMP-1 gene deficiency increases tumor cell sensitivity to chemotherapy-induced apoptosis.
Br J Cancer, 2006, 95: 1114-1120
Durbecq V, Desmed C, Paesmans M, Cardoso F, Di Leo A, Mano M, et al. “Correlation between topoisomerase-IIalpha gene amplification and protein expression in HER-2 amplified breast cancer.” Int J Oncol 2004; 25(5):1473-9]
Ejlertsen B, Mouridsen H T, Jensen M B et al. Improved outcome from substituting methotrexate with epirubicin: results from a randomised comparison of CMF versus CEF in patients with primary breast cancer. Eur J Cancer 2007; 43(5):877-884
Harris et al., op cit; Di Leo A, Gancberg D, Larsimont D, Tanner M, Jar-vinen T, Rouas G, et al. “HER-2 amplification and topoisomerase IIalpha gene aberrations as predictive markers in node-positive breast cancer patients randomly treated either with an anthracycline-based therapy or with cyclophosphamide, methotrexate, and 5-fluorouracil.” Clin Cancer Res 2002; 8(5):1107-16
Jørgensen J T., Nielsen, K. V and Ejlertsen, B, et al. “Pharmacodiagnostics and Targeted Therapies—A Rational Approach for Individualizing Medical Anticancer Therapy in Breast Cancer”: The Oncologist 2007:12:397-405
Knoop A S, Knudsen H, Balslev E, Rasmussen B B, Oyergaard J, Nielsen K V, et al. “Retrospective analysis of topoisomerase Ha amplifications and deletions as predictive markers in primary breast cancer patients randomly assigned to cyclophosphamide, methotrexate, and fluorouracil or cyclophosphamide, epirubicin, and fluorouracil: Danish Breast Cancer Cooperative Group. J Clin Oncol 2005; 23(30):7483-90
Nanna Møller Sørensen, Barry L Dowell, Kent D Stewart, Vibeke Jensen, Lise Larsen, Ulrik Lademann, Gillian Murphy, Hans Jørgen Nielsen, Nils Brünner and Gerard J. Davis. Establishment and Characterization of 7 New Monoclonal Antibodies to Tissue Inhibitor of metalloproteinases-1. Tumor Biology, 2005, 26: 71-80
Mueller R E, Parkes R K, Andrulis I1 O'Malley F P. “Amplification of the TOP2A gene does not predict high levels of topoisomerase Il alpha protein in human breast tumor samples.” Genes Chromosomes Cancer 2004; 39(4):288-97
O'Malley F P., Chia S, Tu D, Shepherd et al., JNCI-2009, 101:644-650
Park K, Kim J, Lim S, Han S. “To-poisomerase ll-alpha (topoll) and HER2 amplification in breast cancers and response to preoperative doxorubicin chemotherapy.” Eur J Cancer 2003; 39(5):631-4
Petit T, Wilt M, Velten M, Millon R, Rodier J F, Borel C, et al. “Comparative value of tumour grade, hormonal receptors, Ki-67, HER-2 and topoisomerase Il alpha status as predictive markers in breast cancer patients treated with neoadjuvant anthracycline-based chemotherapy.” Eur J Cancer 2004; 40(2):205-11
Press M F, Mass R D, Zhou J Y, Sullivan-Halley J, Villalobos I E, Lieberman G, et al. “Association of topoisomerase ll-alpha (TOP2A) gene amplification with responsiveness to anthracy-cline-containing chemotherapy among women with metastatic breast cancer entered in the Herceptin H0648g pivotal clinical trial.” In: ASCO Annual Meeting; 2005; 2005. Abstract No. 9543
Anne Sofie Schrohl, Marion E Meijer-van Gelder, Mads Holten-Andersen, Ib Jarle Christensen, Maxime P Look, Henning T Mouridsen, Nils Brünner and John Foekens. Primary tumor levels of Tissue Inhibitor of Metalloproteinases-1 are predictive of resistance to chemotherapy in patients with metastatic breast cancer. Clin Cancer Res, 2006, 12: 7054-7058
Irene Vejgaard Sorensen, Claus Fenger, Henrik Winther, Niels T Foged, Ulrik Lademann, Nils Brünner and Pernille A Usher. Characterization of Anti-TIMP-1 Monoclonal Antibodies for Immunohistochemical Localization in Formalin-fixed, Paraffin-embedded Tissue. J. Hist. Cytochem, 2006, 54:1075-1086
Sørensen N M, Byström P, Christensen I J, Berglund Å, Nielsen H J, Brünner N, Glimelius B. Plasma Tissue Inhibitor of Metalloproteinases-1 (TIMP-1) predicts objective response and survival in patients with metastatic colorectal cancer receiving first line treatment with irinotecan in combination with 5FU and Folinic acid. Clin Cancer Res, 2007, 13:4117-4122
Tanner M M, Isola J J, Wiklund T, Erikstein B, Kellokumpu-Lehtinen P, Malmstrom P, et al. “Topoisomerase Na gene amplification predicts favorable outcome of tailored and dose-escalated anthracyclin-based adjuvant chemotherapy in HER-2 positive breast cancer. Results from the randomized trial SBG 9401.” In: ASCO Annual Meeting; 2005; 2005. Abstract No. 9518
WO 2005/094863
WO 2007/112746

1. A method for predicting the response to a topoisomerase IIα inhibitor therapy in an individual having cancer, said method comprising the steps of:
a. determining in a sample obtained from said individual, the absence of TIMP-1 protein in tumour cells comprised in said sample or presence of a TIMP-1 DNA aberration in the tumour cells of said sample;
b. determining the presence of any chromosomal DNA aberration in the TOP2A/HER2 amplicon on chromosome 17q21 or aberrant protein expression of a gene comprised in said amplicon;
c. classifying the individual as having a high likelihood of responding to a topoisomerase IIα inhibitor therapy if a chromosomal DNA aberration in the TOP2A/HER2 amplicon on chromosome 17q21 is present or the protein expression of the gene comprised in said amplicon is aberrant in said tumour cells or the tumour cells are absent of TIMP-1 protein or if said tumour cells comprise said TIMP-1 DNA aberration on either or both of the alleles of the TIMP-1 gene; and
d. classifying the individual as having a low likelihood of responding to a topoisomerase IIα inhibitor therapy if no chromosomal DNA aberration in the TOP2A/HER2 amplicon is present or no protein encoded by any gene comprised in said amplicon is aberrantly expressed in the tumour cells and if TIMP-1 protein is present in the tumour cells; if neither of the TIMP-1 alleles comprise said TIMP-1 DNA aberration.

2-42. (canceled)

43. The method according to claim 1, wherein the chromosomal DNA aberration in the TOP2A/HER2 amplicon on chromosome 17q21 is a TOP2A DNA aberration, and the protein expression of the gene comprised in said amplicon is topoisomerase IIa expression.

44. The method according to claim 1, wherein the chromosomal DNA aberration in the TOP2A/HER2 amplicon on chromosome 17q21 is a HER2 DNA aberration, and the protein expression of the gene comprised in said amplicon is ErbB2 expression.

45. A method for predicting the response to a topoisomerase IIα inhibitor therapy in an individual having cancer, said method comprising the steps of:
a. determining in a sample obtained from said individual, the absence of TIMP-1 protein in tumour cells comprised in said sample;
b. determining the presence of any TOP2A DNA aberration in the tumour cells of said sample;
c. classifying the individual as having a high likelihood of responding to a topoisomerase IIα inhibitor therapy if a TOP2A DNA aberration is present or if the tumour cells are absent of TIMP-1 protein; and
d. classifying the individual as having a low likelihood of responding to a topoisomerase IIα inhibitor therapy if no TOP2A DNA aberration is present and if TIMP-1 protein is present in the tumour cells.

46. The method according to claim 43, wherein the TOP2A gene aberration is selected from the group consisting of TOP2A DNA amplification, TOP2A DNA deletion, TOP2A gene point mutation, TOP2A DNA translocation, and epigenetic modifications of the TOP2A DNA or a combination thereof.

47. The method according to claim 43, wherein topoisomerase IIα protein is more than 2 fold over-expressed relative to a reference sample.

48. The method according to claim 43, wherein TOP2A gene is more than 2 fold amplified relative to a reference sample.

49. The method according to claim 44, wherein the HER2 gene aberration is selected from the group consisting of HER2 gene amplification, HER2 DNA deletion, HER2 gene point mutations, HER2 DNA translocations, and epigenetic modifications of the HER2 DNA or a combination thereof.

50. The method according to claim 44, wherein ErbB2 protein is more than 2 fold over-expressed relative to a control sample.

51. The method according to claim 44, wherein HER2 gene is more than 2 fold amplified relative to a control sample.

52. The method according to claim 1, wherein TIMP-1 gene is more than 2 fold amplified relative to a control sample.

53. The method according to claim 44, wherein the any HER2 DNA aberration or an increase in ErbB2 protein in the tumour cells correlate with aberrant HER2 mRNA levels in the tumour cells of said sample.

54. The method according to claim 1, wherein the tumour cells comprise at least one TIMP-1 DNA aberration selected from the group consisting of a deletion of one of the TIMP-1 alleles, a deletion of both of the TIMP-1 alleles, a partial deletion of one of the TIMP-1 alleles, a partial deletion of both of the TIMP-1 alleles, TIMP-1 DNA point mutations, TIMP-1 DNA inversion, TIMP-1 DNA translocation, and epigenetic modifications of the TIMP-1 DNA or a combination thereof.

55. The method according to claim 1, wherein the level of DNA gene aberration is determined by DNA measurement.

56. The method according to claim 1, wherein the cancer is selected from the group consisting of breast cancer, sarcomas, ovarian cancer, and non small cell lung cancer.

57. The method according to claim 1, wherein said sample is selected from the group consisting of a tumour tissue sample, a blood sample, a plasma sample, a serum sample, a urine sample, a faeces sample, a saliva sample, and a sample of serous liquid from the thoracic or abdominal cavity or a combination thereof.

58. The method according to claim 1, wherein the likelihood of responding to a topoisomerase IIα inhibitor therapy is determined by a hazard ratio.

59. A method of treating cancer in an individual comprising:
a. predicting the response to an topoisomerase IIα inhibitor therapy according to any of the preceeding claims;
b. selecting an topoisomerase Iha inhibitor therapy to which said individual has a high likelihood of responding to; and
c. subjecting said individual to said topoisomerase IIα inhibitor therapy.

60. The method according to claim 59, wherein the topoisomerase IIα inhibitor is a anthracyclines selected from the group consisting of but not limited to 4-Epirubricin, Daunorubicin, Daunorubicin (liposomal), Doxorubicin, Doxorubicin (liposomal), Epirubicin, Idarubicin, and Mitoxantrone, or a combination thereof.

61. A kit for predicting the response to a topoisomerase IIα inhibitor therapy comprising:
a. reagents suitable for the determination of a chromosomal DNA aberration in the TOP2A/HER2 amplicon; and
b. reagents suitable for the determination of a TIMP-1 DNA aberration or determining the level of a TIMP-1 protein in a biological sample.
